(19) United States
(12) Patent Application Publication
(10) Pub. No.: US 2022/0339118 A1
(43) Pub. Date: Oct. 27, 2022

(54) REGENERATIVE BIOACTIVE SUSPENSION DERIVED FROM FRESHLY DISAGGREGATED TISSUE AND METHODS OF USE IN CLINICAL THERAPIES

(71) Applicant: AVITA Medical, Inc., Valencia, CA (US)

(72) Inventors: Katie BUSH, Marblehead, MA (US); Amelia Ruth HOPKIN, Aliso Viejo, CA (US); Briana Rachel LEE ALTMAN, Mission Viejo, CA (US); Lauren Heard MANGUM, Fishers, IN (US); Kurinji PANDIYAN, Sherman Oaks, CA (US); Andrew Perry QUICK, Valencia, CA (US); Sohail JAHID, Garden Grove, CA (US); Ghaidaa KASHGARI, Irvine, CA (US)

(21) Appl. No.: 17/727,213

(22) Filed: Apr. 22, 2022

Related U.S. Application Data
(63) Continuation of application No. 17/724,986, filed on Apr. 20, 2022.
(60) Provisional application No. 63/180,505, filed on Apr. 27, 2021.

(51) Int. Cl. A61K 9/70; A61P 17/02; A61K 38/18; A61K 35/12

(52) U.S. Cl. CPC A61K 9/7015; A61P 17/02; A61K 38/18; A61K 35/12

(57) ABSTRACT

A bioactive suspension derived from freshly disaggregated tissue is provided, as well as related methods of formulation and use. The bioactive suspension may comprise a cell-free supernate derived from epidermal and dermal tissue that has been enzymatically and mechanically disaggregated, then separated, and which may contain tissue regeneration factors known to speed healing. The bioactive suspension may further comprise genetically-modified treatment cells, wild type cells, or both, and may be combined with one or more scaffolding elements to form a bioactive suspension combination product suitable for treatment of a cutaneous defect. Synthetic bioactive suspensions and bioactive suspension combination products are also provided.

### RELATED APPLICATIONS

This application is a continuation of U.S. patent application Ser. No. 17/724,986 filed Apr. 20, 2022, which claims the benefit of U.S. Provisional Patent Application No. 63/180,505 filed Apr. 27, 2021, the disclosure of each of which is incorporated herein by reference in its entirety.

### TECHNICAL FIELD

The present invention generally relates to the field of regenerative medicine.

### BACKGROUND

Within the field of regenerative medicine, many therapies are available to speed healing of a wound, most having their benefits and drawbacks.

For example, allogeneic skin grafts are used in the care of burn patients. They are commercially available as cryopreserved and glycerol-preserved skin sheets and function as biologic dressing, providing temporary wound coverage, preparing the wound bed for definitive wound closure by secondary intention or surgical means (autologous skin graft). They are not used as permanent skin substitutes as the skin allograft is rejected by the recipient following recognition of graft antigens leading to the cellular destruction of the graft.

Allogeneic dermal matrices (ADMs) are used in the care of cutaneous defects to provide temporary closure and assist in the preparation of the wound bed for definitive closure. These products are decellularized to remove native cells and consist of an extracellular matrix composed predominately of collagenous proteins interspersed in a basketweave pattern. Following application in a wound bed, the patient's cells infiltrate the matrix, repopulating and revascularizing it. The material is integrated and is not removed. For permanent closure, an autologous skin graft can be used to achieve immediate closure, or the wound can be left to heal by secondary intention.

Allogeneic cell-based bioengineered skin substitutes are used for the treatment of cutaneous defects. Living cells are combined with biomaterials and are grown in organotypic culturing systems to produce living skin substitutes. When placed in the wound bed, the allogeneic cells within the construct actively secrete growth factors and cytokines to promote healing. The cells within the skin substitutes do not contain cells with MHC class II antigens, greatly reducing the occurrence of allograft antigen presentation.

Human amnion/chorion membrane allografts are used in the care of cutaneous defects including acute and chronic wounds. Products that are dehydrated retain an extracellular matrix and regulatory proteins that are introduced into the wound environment to stimulate healing. Cryopreserved products are comprised of an extracellular matrix, growth factors, and cells (fibroblasts, mesenchymal stem cells, epithelial cells) that elicit healing responses when placed into the wound environment.

Relatedly, it is known that bioactive molecules regulate signaling pathways which are critical for the communication between cells during development, homeostasis, and repair. They are involved in stimulating cellular processes including protein synthesis and deposition, cell proliferation, migration, differentiation, and cell survival. These molecules include proteins, cytokines, lipids, and exosomes, as well as cell-free nucleic acids. Presenting a proper bioactive environment to cells for transplantation or to host cells can direct desired processes and optimize functional outcomes.

Based on therapeutic application, there are different approaches to provide bioactive cellular signals to the body:

### Cell-Based Therapy

Cell-based therapy involves the transfer of viable cells with relevant function into the patient. These cells may be the patient's own cells or cells from a donor that have been cultured or modified to alleviate a medical condition. When grafting or transplanting the cells, it is essential that the delivery environment enables the cells to maintain viability, support engraftment, and provides signaling cues to direct functionality upon implantation. The faster a robust tissue can be formed, the sooner the clinical benefit can be realized.

There is no state of the art with respect to cell-based therapy, as cell-based therapy is a nascent field and advances are needed to optimize the delivery of cells for tissue regeneration as applications increase.

### Wound Healing

For wound healing applications, living skin substitutes are grown from allogeneic tissue sources and placed on a prepared wound bed. In these examples, the cells actively secrete growth factors and cytokines to promote healing.

### Rejuvenation

For cosmeceutical applications, cell conditioned media is used to activate cells in vivo. This approach involves the culture of cells and removal of the media.

However, the general state of the art does not provide for a bioactive suspension derived from freshly harvested and disaggregated skin tissue. Instead, these therapies rely on cultured cells or decellularized materials (amnionic tissue).

What is needed is a bioactive suspension useful as a standalone therapeutic, that would accelerate wound healing or that could be used as an agent for delivery of cellular therapy. Preferably, the bioactive suspension is useful as either an autologous or allogeneic standalone therapeutic.

### SUMMARY

The invention described is a bioactive suspension derived from freshly disaggregated tissue. In some embodiments, the bioactive suspension comprises a supernate that contains tissue regeneration factors: soluble protein factors inclusive of growth factors, cytokines, danger associated molecular patterns (DAMPs), proteases, exosomes, cell-free nucleic acids, other signaling molecules, cell fragments, and other cellular products released by tissue cells when the tissue is disaggregated. In some embodiments, one or more tissue regeneration factors act as signaling cues to enhance cellular functions and promote robust tissue formation.

A method of formulation of a bioactive suspension may comprise harvesting a tissue sample, disaggregating the tissue sample, which in some embodiments may comprise enzymatic disaggregation, mechanical disaggregation, or both, then allowing the disaggregated tissue sample to rest in solution for an effective amount of time, followed by separating out the cells of the disaggregated tissue sample from the solution, and then obtaining a supernate comprising the bioactive suspension.

In some embodiments, a method of formulation of a bioactive suspension may further comprise the step of freezing the bioactive suspension, such as by way of nonlimiting example, at −80 C, then thawing the bioactive suspension prior to use.

In some embodiments, a method of formulation of a bioactive suspension may further comprise the step of adding treatment cells. In such embodiments, the treatment cells may comprise cells obtained from the freshly disaggregated tissue used to obtain the bioactive suspension, cultured cells, gene-edited cells, induced pluripotent stem cells (iPSCs), forward-differentiated cells, or another type of cell. Related methods of use are also provided.

In some embodiments, a method of formulation of a bioactive suspension combination product may comprise the additional step of combining a bioactive suspension made according to one or more methods disclosed herein with one or more scaffolding elements, such as but not limited to a skin graft, matrix, hydrogel, bioprinted gel, bioprinted tissue, or dressing.

A bioactive suspension composition may comprise a bioactive suspension composition produced by one or more methods disclosed herein. In some embodiments, a bioactive suspension composition may comprise a base solution supplemented by tissue regeneration factors, and prepared according to techniques known in the art. As well, in some embodiments, a bioactive suspension composition may comprise a bioactive composition prepared according to one or more methods disclosed herein and that contains one or more treatment cells.

A bioactive suspension combination product may comprise a bioactive suspension prepared according to one or more methods disclosed herein and combined with one or more matrix elements. It is contemplated that in some embodiments, a bioactive therapeutic suspension may further comprise treatment cells.

In some embodiments, the tissue sample may be autologous relative to the person or organism to whom the bioactive suspension is applied (“patient”). In some embodiments, the tissue sample may be allogeneic relative to the patient. In some embodiments, the tissue sample may be xenogeneic relative to the patient.

In one or more embodiments of the bioactive suspension, the bioactive suspension may be applied to a treatment area of a patient using one or more methods of application disclosed herein.

It is contemplated that embodiments of the present bioactive suspension may be formulated for use as a standalone therapeutic.

### BRIEF DESCRIPTION OF THE DRAWINGS

FIG. 1 is a flowchart illustrating an exemplary variation of a method for preparing a bioactive suspension for use as a standalone therapeutic.

FIG. 2 is a flowchart illustrating an exemplary variation of a method for preparing a bioactive suspension for use as a delivery agent for a cell-based therapy.

FIG. 3 is a flowchart illustrating an exemplary variation of a method for preparing a bioactive suspension for use in combination with the addition of an efficacious element.

FIG. 4 depicts two sets of tissue cross sections showing the effects of seeding treatment areas with and without the bioactive suspension at a 1E6 concentration of cells seeded.

FIG. 5 depicts two sets of tissue cross sections showing the effects of seeding treatment areas with and without the bioactive suspension at a 0.5E6 concentration of cells seeded.

FIG. 6 depicts a graph showing experimental day 6 re-epithelialization results of wounds treated with a meshed split-thickness skin graft (mSTSG) and an embodiment of the bioactive suspension compared to wounds treated with the mSTSG alone.

FIG. 7 depicts a graph showing Ki67 staining results of wounds treated with a mSTSG and an embodiment of the bioactive suspension compared to wounds treated with the mSTSG alone.

FIG. 8 depicts a graph showing transepidermal water loss (TEWL) results of wounds treated with a mSTSG and an embodiment of the bioactive suspension compared to wounds treated with the mSTSG alone.

FIG. 9A depicts a graph showing protein concentrations at 1:80 for the bioactive suspension over time.

FIG. 9B depicts a graph showing protein concentrations at 1:5 and 1:80 for the bioactive suspension over time.

FIG. 10A depicts a graph showing expression levels of the protein decorin in the wound bed of wounds treated with a mSTSG and an embodiment of the bioactive suspension compared to wounds treated with mSTSG alone. The legend in FIG. 10C applies to FIG. 10A, namely that circles indicate mSTSG and triangles indicate mSTSG+bioactive suspension in at least one embodiment.

FIG. 10B depicts a graph showing expression levels of the protein GASP-1 in the wound bed of wounds treated with a mSTSG and an embodiment of the bioactive suspension compared to wounds treated with mSTSG alone. The legend in FIG. 10C applies to FIG. 10B, namely that circles indicate mSTSG and triangles indicate mSTSG+bioactive suspension in at least one embodiment.

FIG. 10C depicts a graph showing expression levels of the protein TWEAK R in the wound bed of wounds treated with a mSTSG and an embodiment of the bioactive suspension compared to wounds treated with mSTSG alone.

### DETAILED DESCRIPTION

Skin cells, when stress-induced such as by mechanical or enzymatic disaggregation or by freezing, secrete a multitude of tissue regeneration factors known to stimulate, accelerate, mediate, and/or sustain tissue regeneration. Tissue regeneration factors provide broad mitogenic, immunomodulatory, pro-inflammatory, anti-inflammatory, anti-microbial, and damage associated molecular patterns (DAMPs) important in the wound healing cascade. It is now shown herein for the first time that in some embodiments, tissue regeneration factors within the present bioactive suspension not only stimulate the regeneration of tissue at the edge of a wound bed, that they also stimulate tissue regeneration across the entirety of a wound bed, enabling faster healing, evenly dispersed tissue regrowth, and an increase in transplanted cell viability.

By utilizing freshly harvested tissue rather than cultured cells as the starting material from which the bioactive suspension is derived, and therefore obviating the required conditioned media elements and known cellular changes associated with cultured cells, the present bioactive suspension shows surprising efficacy as both a standalone therapeutic and an agent for delivery of cell therapies.

Moreover, the present disclosure presents the surprising information that freshly disaggregated cells release significant amounts of tissue regeneration factors, sometimes in as little as 30 minutes. In some embodiments, this amount of tissue regeneration factors may be comparable to the amount of tissue regeneration factors released by cultured cells over a span of several days.

As well, this approach departs from conventional wisdom in the field of regenerative medicine. As discussed above, it is generally thought that the presence of live cells that are able to proliferate is vital for tissue regeneration in a skin wound. For the first time, the present disclosure presents data tending to show that embodiments of the cell-free bioactive suspension stimulate skin tissue regeneration as well or in some instances better than regenerative therapies that include cells.

By using freshly harvested and disaggregated skin tissue, then separating out the cells, the present bioactive suspension provides one or more advantages over the use of other starting tissue elements, such as but not limited to cultured cells, engineered tissue, adipose tissue, amniotic tissue allografts, amnionic fluid allografts, other placental products or therapies, decellularized dermis, decellularized fluids, acellular tissues or fluids, or other such therapies, as the starting materials or active components. By way of illustration and not limitation, the present bioactive suspension may provide one or more tissue regeneration factors not found in the other therapies, may involve a simpler method of production, may be more efficacious, or provide another benefit. In one or more embodiments, the tissue may comprise frozen then thawed tissue, which may then be disaggregated.

In some embodiments, the bioactive suspension may further comprise treatment cells and thus may comprise a delivery agent for one or more cell-based therapies. In such a variation, the bioactive suspension may be used to deliver treatment cells to a treatment area on a patient and enhance the desired outcome. By way of non-limiting examples, the treatment cells may comprise autologous cells, allogeneic cells, xenogeneic cells, or a combination of two or more of autologous cells, allogeneic cells, and xenogeneic cells. Moreover, the treatment cells may comprise gene-edited cells, non-gene-edited cells (“wild type” cells), or both gene-edited cells and non-gene-edited cells. The treatment cells may comprise living cells, nonliving cells, or both living and nonliving cells. In one or more embodiments, the bioactive suspension comprises (i) the bioactive suspension; (ii) treatment cells; and (iii) wild type cells.

In some embodiments, the bioactive suspension may comprise a stand-alone therapeutic. In such variations, one or more components of the bioactive suspension, including but not limited to one or more tissue regeneration factors, may initiate an in vivo cellular activation of one or more host cells of a patient. Exemplary applications of one or more embodiments of the bioactive suspension when formulated or used as a stand-alone therapeutic include treatment for chronic wounds, partial-thickness burn injuries, full-thickness burn injuries, skin rejuvenation, other epithelial tissue repair or rejuvenation, and other applications.

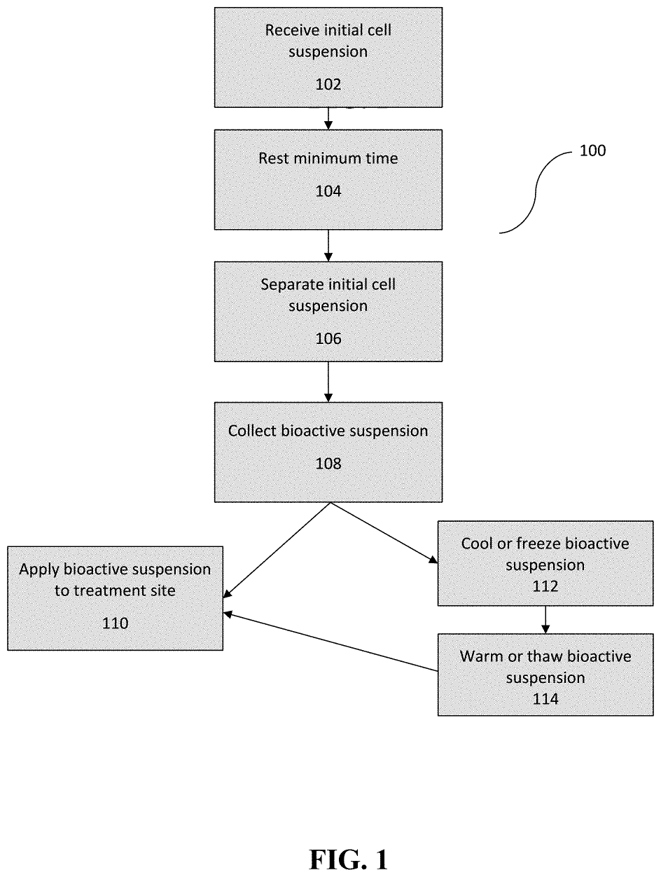

FIG. 1 is a flowchart illustrating an exemplary variation of a method 100 for preparing a bioactive suspension for use as a standalone therapeutic. At 102, the method 100 may include receiving an initial cell suspension. At 104, the initial cell suspension may be allowed to rest for a minimum time. At 106, the initial cell suspension may be separated to remove the cells. The bioactive suspension may then be collected 108 applied to a treatment site 110, or the bioactive suspension may be cooled or frozen 112. If the bioactive suspension is cooled or frozen 112, when ready to be delivered to a patient, the bioactive suspension may be warmed or thawed 114, then applied to a treatment site 110.

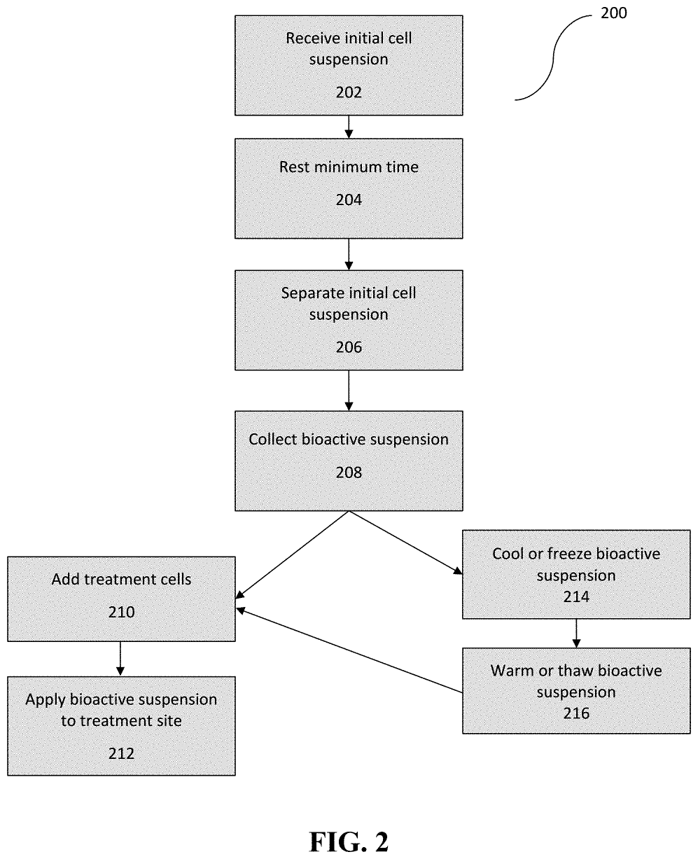

FIG. 2 is a flowchart illustrating an exemplary variation of a method 200 for preparing a bioactive suspension for use as a delivery agent for a cell-based therapy. At 202, the method 200 may include receiving an initial cell suspension. At 204, the initial cell suspension may be allowed to rest for a minimum of thirty minutes. At 206, the initial cell suspension may be separated to remove the cells. At 208, the bioactive suspension may be collected. Thereafter, in some embodiments, treatment cells may be added 210 and then the resulting bioactive suspension may be applied to a treatment site 212. Alternatively, following collection of the bioactive suspension 208, the bioactive suspension may be cooled or frozen 214, then warmed or thawed 216, after which one or more treatment cells may be added to the cooled-then-warmed or frozen-then-thawed bioactive suspension 210 and then the resulting bioactive suspension may be applied to a treatment site 212.

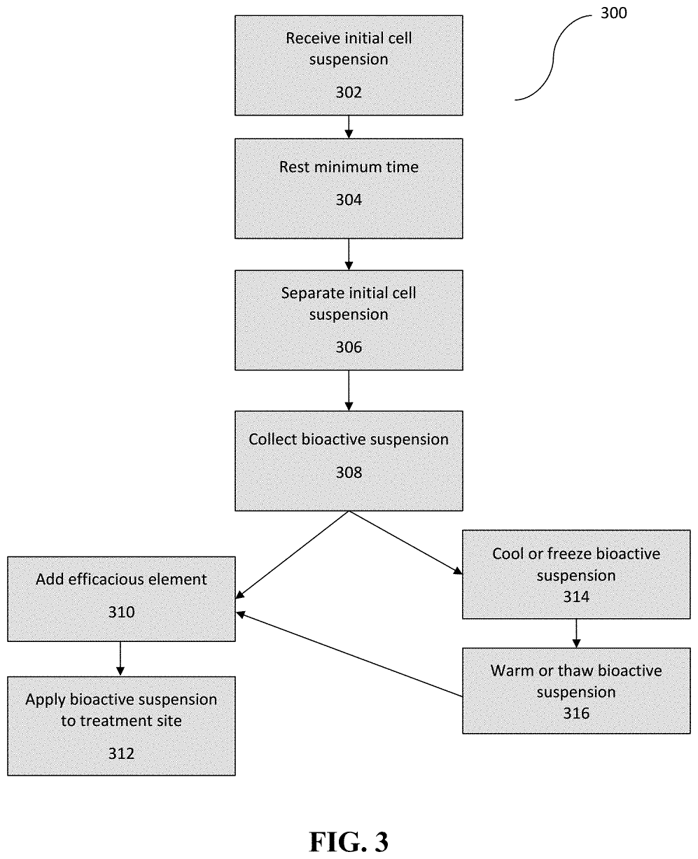

FIG. 3 is a flowchart illustrating an exemplary variation of a method 300 for preparing a bioactive suspension for use in combination with the addition of an efficacious element. At 302, the method 300 may include receiving an initial cell suspension. At 304, the initial cell suspension may be allowed to rest for a minimum of thirty minutes. At 306, the initial cell suspension may be separated to remove the cells. At 308, the bioactive suspension may be collected. Thereafter, in some embodiments, at least one efficacious element may be added 310 and then the resulting bioactive suspension may be applied to a treatment site 312. Alternatively, following collection of the bioactive suspension 308, the bioactive suspension may be cooled or frozen 314, then warmed or thawed 316, after which at least one efficacious element may be added to the cooled-then-warmed or frozen-then-thawed bioactive suspension 310 and then the resulting bioactive suspension may be applied to a treatment site 312.

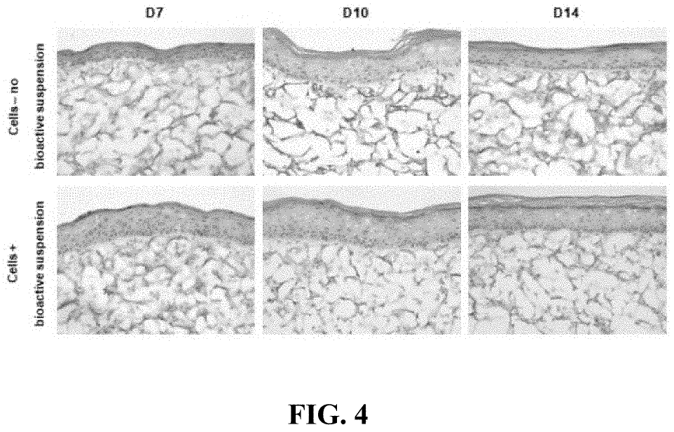

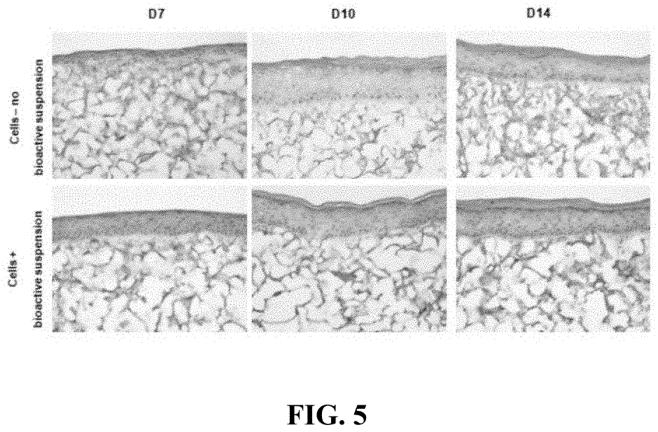

The bioactive suspension provides several advantages to these traditional cell delivery techniques. For example, as can be seen in FIG. 4 and FIG. 5, the bioactive suspension promotes faster regeneration of tissue than traditional cell delivery techniques.

More specifically, as may be seen in FIG. 4 and FIG. 5, cell seeding treatments that include the bioactive suspension have been shown to stimulate greater tissue regeneration than cell seeding treatments without the bioactive suspension. As can also be seen in FIG. 4 and FIG. 5, the bioactive suspension enables cell seeding treatments to be effective even when the number of cells seeded is greatly reduced. This indicates that the tissue regeneration factors in the bioactive suspension have new and unexpected effects in cell therapy treatments.

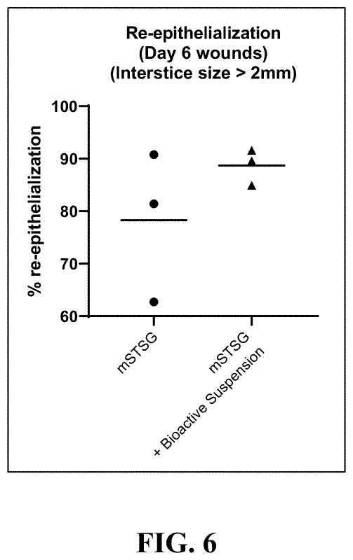

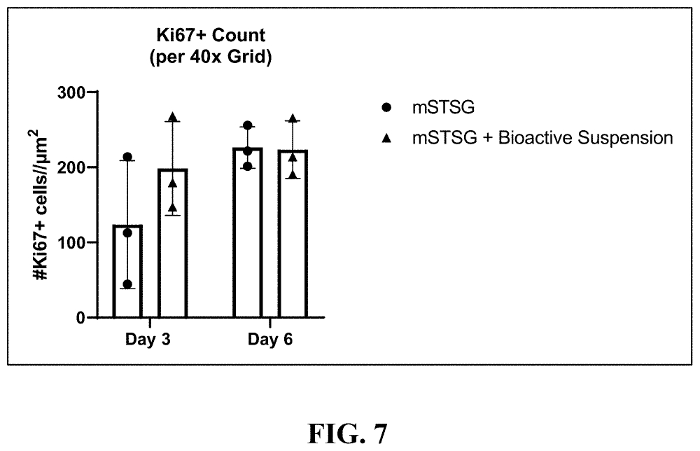

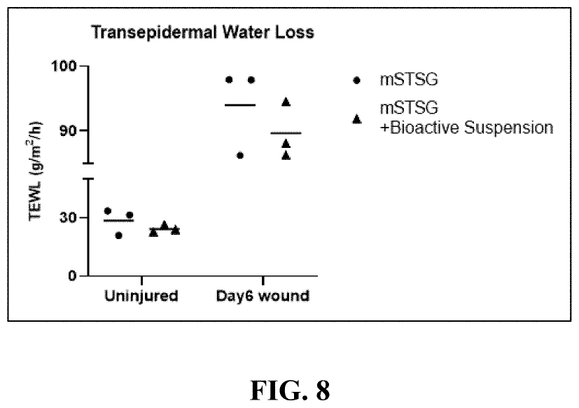

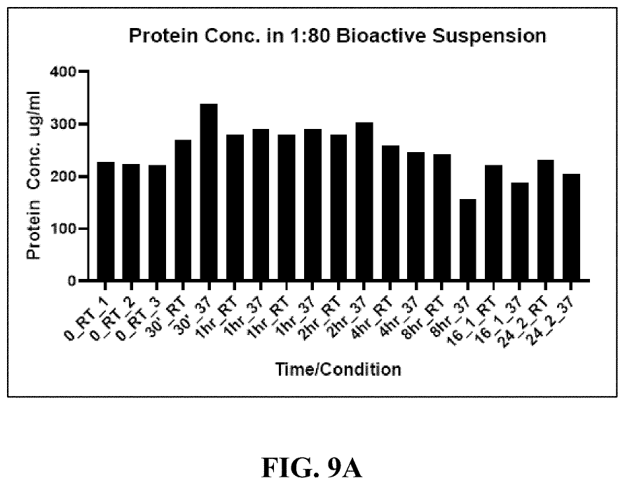

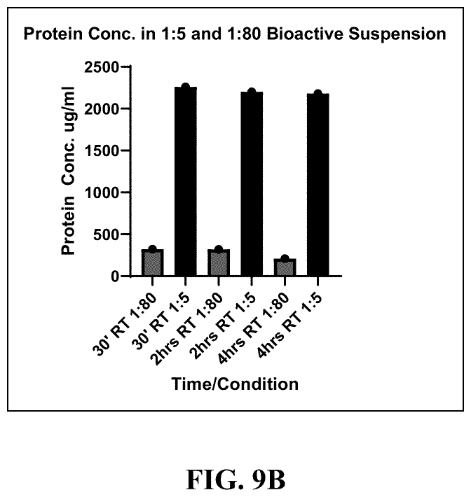

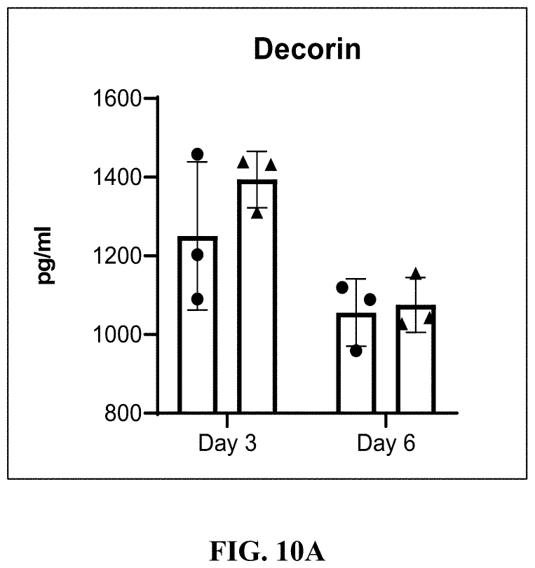

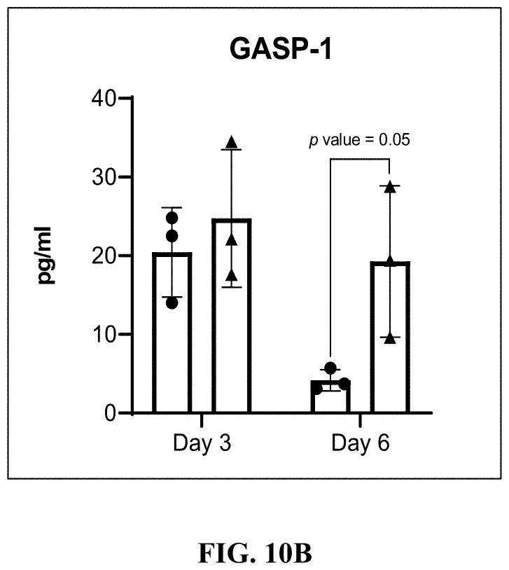

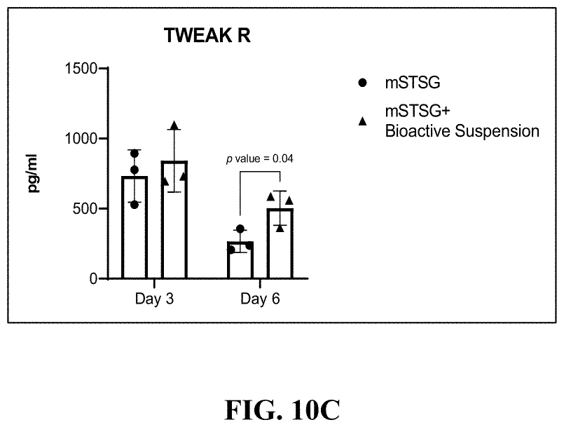

FIG. 6, FIG. 7, FIG. 8, FIG. 9A, FIG. 9B, FIG. 10A, FIG. 10B, and FIG. 10C are discussed below in connection with one or more experiments.

### Method of Preparing the Bioactive Suspension

A method of preparing a bioactive suspension may comprise (1) obtaining a tissue sample, (2) subjecting the tissue sample to disaggregation, [which in some embodiments may comprise (a) enzymatic disaggregation followed by rinsing residual enzyme; (b) mechanical disaggregation; or (c) enzymatic disaggregation followed by mechanical disaggregation], (3) adding additional buffer to obtain a cell suspension, (4) allowing the cell suspension to rest for an effective amount of time, (5) separating the cells of the cell suspension out of the suspension to obtain a bioactive suspension, (6) optionally, freezing and then thawing the bioactive suspension, (7) optionally, adding one or more populations of treatment cells to the bioactive suspension to obtain a bioactive suspension configured for use as a cell therapy delivery agent, (8) optionally, adding one or more tissue regeneration factors to the bioactive suspension, and (9) optionally, adding one or more efficacious elements to the bioactive suspension. It will be evident to one of skill in the art that one or more of the steps may be omitted or done in a different sequence, and that one or more of steps 6-9 may be optionally omitted.

The tissue sample may be harvested from a living person, a recently deceased person, or a non-human source, such as a plant, animal, insect, or microorganism. In some embodiments, the tissue sample may comprise tissue harvested from more than one source. In some embodiments, the tissue sample may be autologous, allogeneic, or xenogeneic relative to the patient, or a combination of two or more tissue samples that are autologous, allogeneic, or xenogeneic relative to the patient. In some embodiments, the tissue sample may comprise tissue that has been frozen then thawed prior to disaggregation.

The tissue sample may in some embodiments comprise skin tissue. In some embodiments, the skin tissue may comprise one or more of human skin, a split-thickness human skin sample, a full thickness human skin sample, human skin epidermis, human skin dermis, human skin epidermal-dermal junction tissue, human mesenchymal stem cells, human adipose tissue, non-human epidermis, non-human dermis, non-human epidermal-dermal junction tissue, non-human mesenchymal stem cells, and non-human adipose tissue.

Examples of non-human tissue sources include but are not limited to urodele skin, adipose tissue and mesenchymal stem cells; anuran skin, adipose tissue and mesenchymal stem cells; porcine skin, adipose tissue and mesenchymal stem cells; bovine skin, adipose tissue and mesenchymal stem cells; murine skin, adipose tissue and mesenchymal stem cells; fish such as but not limited to goldfish, zebrafish, catfish, trout, tilapia, aglomerular toadfish, Physiculus cyanostrophus, genus Cyclothone, family Poeciliidae (order Cyprinodontiformes), superclass Osteichthyes skin, class Sarcopterygii, or class Actinopterygii, adipose tissue and mesenchymal stem cells; invertebrate such as but not limited to Hydra sp., Platyhelminthes phylum, or Planaria sp., skin, adipose tissue and mesenchymal stem cells; and marsupial skin, adipose tissue and mesenchymal stem cells. In preferred embodiments, no cellular culture or expansion is required to obtain the tissue sample. In some embodiments, the tissue sample may comprise 3D printed skin. In some embodiments, the tissue sample may comprise skin equivalents.

The thickness of the tissue sample may vary. As mentioned previously, the tissue sample may in some embodiments comprise a split thickness skin tissue sample or a full thickness skin tissue sample. As used herein, split-thickness skin tissue samples may be grouped according to their thickness into thin split-thickness skin tissue samples (0.15 mm to 0.3 mm thick), intermediate split-thickness skin tissue samples (0.3 mm to 0.45 mm thick), and thick split-thickness skin tissue samples (0.45 to 0.6 mm thick). As used herein, a full thickness skin tissue sample may comprise full-thickness epidermis and partial-thickness dermis, and may range in thickness between 0.06 mm and 5.08 mm thick.

Unless otherwise stated or context implies otherwise, the term “tissue sample” as used herein is intended to mean a split thickness skin tissue sample. The term “split thickness” skin tissue sample as used herein comprises the range of 0.15 mm through 0.6 mm thick.

Likewise, the term “tissue cells” refers to those cells that were once part and parcel with the tissue sample, but have been disaggregated according to one or more steps detailed herein, such as but not limited to enzymatic disaggregation or mechanical disaggregation. Tissue cells may comprise but are not limited to one or more type of cell from the list comprising keratinocytes inclusive of epidermal stem cells, proliferating keratinocytes, stem cells, progenitor cells, basal keratinocytes, activated keratinocytes, and suprabasal keratinocytes, mesenchymal stem cells, immune cells, fibroblasts, endothelial cells, and melanocytes, any type of epidermal cell, any type of epithelial cell, or a cell from at least one area of an epidermal-dermal junction, said area comprising but not limited to the basal cell plasma membrane, lamina lucida, basal lamina, or sub-basal lamina fibrous component area.

In some preferred embodiments, the tissue sample's thickness may be in the range of 0.15 mm to 0.20 mm (0.006 inches-0.008 inches). In some variations, the tissue sample's thickness may be 0.15 mm to 0.25 mm (0.006-0.01 inches). In some variations, the tissue sample's thickness may be 0.2 mm to 0.4 mm (0.007-0.016 inches). In some variations, the tissue sample's thickness may be 0.025 mm to 0.152 mm (0.001-0.006 inches). In some variations, the tissue sample's thickness may be >0.01 inches. The tissue harvested may, in some embodiments, comprise skin, epithelial tissue, internal organ tissue, bone, or other tissue.

In some variations, the tissue sample may be harvested using a dermatome, knife, scalpel, or other suitable instrument. By way of illustration and not limitation, exemplary dermatomes include a Blair/Brown knife, a Humby knife, a Braithwaite knife, a Watson knife, a Cobbett knife, a Goulian/Weck knife, a Silver knife, a Padgett dermatome, a Reese dermatome, a Brown dermatome, a Zimmer® dermatome, a Castroviejo dermatome, an air dermatome, other electric dermatome, other drum dermatome, other knife dermatome, or other dermatome.

The tissue sample may then be subjected to disaggregation. In some embodiments, enzymatic disaggregation may comprise the disaggregation step. In some embodiments, mechanical disaggregation may comprise the disaggregation step. In some embodiments, enzymatic disaggregation followed by mechanical disaggregation may comprise the disaggregation step. Alternatively, in some embodiments, mechanical disaggregation followed by enzymatic disaggregation may comprise the disaggregation step. In preferred embodiments, the disaggregation step may comprise enzymatic disaggregation followed by mechanical disaggregation.

In embodiments utilizing enzymatic disaggregation, the enzymatic disaggregation may comprise: (i) obtaining an enzyme solution, (ii) warming the enzyme solution to an effective temperature, and (iii) submerging the tissue sample in the warmed enzyme solution for an effective time period.

The enzyme used to enzymatically disaggregate the tissue sample may comprise, but is not limited to, examples such as trypsin, dispase, collagenase, trypsin-EDTA, thermolysin, pronase, hyaluronidase, elastase, papain and pancreatin. In some variations, one or more enzymes such as trypsin, dispase, collagenase, trypsin-EDTA, thermolysin, pronase, hyaluronidase, elastase, papain and pancreatin may be traditionally sourced, such as for example via fermentation, or may be recombinant- or animal-derived. In some variations, an enzyme solution may be formed by mixing lyophilized enzyme with an appropriate volume of fluid (e.g., water). When the enzyme used is trypsin, the enzyme solution is preferably free of calcium and magnesium.

In some variations, the enzyme solution may have an amount of enzyme between about 0.05% and about 5% per volume of solution, between about 0.1% and about 5% per volume of solution, between about 0.25% and about 2.5% per volume of solution, or about 0.5% enzyme per volume of solution.

In some variations, the enzyme solution may be heated to a target temperature (e.g., about 20° C., between about 30° C. and about 37° C., between about 33° C. and about 37° C., or about 37° C.).

For instance, the enzyme solution may be heated by placing the enzyme solution into a heating well, or by using any suitable heating mechanism. In some variations, the enzyme solution may be heated for a suitable period of time until the target temperature is reached. For example, the period of heating time may range from thirty seconds to five minutes. In some embodiments, the enzyme mixture may reach its target temperature in under three minutes of heating. Once heated to the suitable target temperature, the warmed enzyme solution may be suitable for use in processing a tissue sample.

In some variations, a kit similar to that described in U.S. Pat. No. 9,029,140 and/or U.S. patent application Ser. No. 16/935,977, both of which are incorporated by reference herein, may provide resources to prepare a suitable enzyme solution. For example, a kit may provide an enzyme vial of lyophilized enzyme, a vial of sterile water, and/or appropriate measurement or manipulation instruments. A diaphragm of the enzyme vial may be optionally wiped with sterile alcohol wipe and allowed to dry. A syringe may be inserted into an interior of the water vial and an appropriate volume of water may be drawn from the water vial into the syringe. The volume of water may then be injected from the syringe into the interior of the enzyme vial and mixed gently (e.g., without shaking to avoid foaming) until the enzyme is dissolved in the water to form an enzyme solution. The enzyme solution may be drawn back into the syringe for distribution into a heating well or other suitable heating mechanism.

In some variations, the tissue sample may be submerged in the heated enzyme solution (e.g., by submerging the tissue sample in a heating well into which the enzyme solution was dispensed) such that the enzyme solution may break down protein-protein interactions. For example, the tissue sample may be submerged in the enzyme solution for a suitable incubation period, such as but not limited to 1-60 minutes. In some embodiments, the period of enzymatic disaggregation time may comprise at least one minute, sixty minutes or longer, sixty minutes or less, 1-15 minutes, 15-20 minutes, 5-30 minutes, or any other time combination therein. Thinner tissue samples may be submerged in the enzyme solution for 15-30 minutes, whereas thicker tissue samples may be submerged in the enzyme solution for a longer period of time (e.g., up to 60 minutes or longer). In preferred embodiments, the enzymatic disaggregation time may comprise 30 minutes. In preferred embodiments, the enzymatic disaggregation time may comprise approximately 30 minutes. In some variations, thicker tissue samples may be submerged in an enzyme solution with a higher concentration of the enzyme.

The tissue sample may then be contacted with an effective amount of buffer solution, in this case an amount of buffer solution sufficient to substantially deactivate the enzyme's activity. The addition of buffer solution at this step may in some embodiments, result in the production of a cell suspension.

In some variations, the buffer solution may be free of serum xenogeneic to the patient. In such situations, the buffer solution may rinse off, and in some embodiments also deactivate, any residual enzyme solution on the skin sample. In some variations, the buffer may have the characteristics of being (i) free of at least xenogeneic serum, (ii) capable of maintaining the viability of the cells until applied to a patient, and (iii) suitable for direct application to a region on a patient. The buffer may be anything from a simple salt solution to a more complex solution. In some variations, the buffer may be free of all serum but may contain various salts that resemble the substances found in body fluids (e.g., physiological saline). Phosphate or other non-toxic substances may also be used as buffer in order to maintain the pH at approximately physiological levels. Examples of a suitable buffer solution include but are not limited to Hartmann's solution, calcium and magnesium ion free phosphate buffered saline, compound sodium lactate buffer, Lactated Ringer's solution, cell culture media, any combination of such buffers, and any other suitable buffer or combination of buffers known in the art.

After rinsing the tissue sample with buffer, optionally, a test scrape for cell disaggregation may be performed. The test scrape may comprise gently scraping an epidermis edge of the sample with a scalpel to test whether tissue cells are easily removed. To illustrate, the skin sample may be removed from the heated enzyme solution and placed dermal side down on an appropriate surface, such as with sterile forceps or scalpel. The epidermis edge of the skin sample may be scraped gently with the scalpel to test if the tissue sample cells disaggregate (e.g., if the epidermal cells separate easily) into tissue cells. If the tissue sample disaggregates into cells, the scraping may be stopped. If the tissue sample does not disaggregate into cells, the tissue sample may be returned to the heated enzyme solution for a period of time (e.g., about five to ten minutes), and then removed for additional test scraping to determine whether the tissue sample disaggregates into cells.

Remaining with the step of disaggregation, in some embodiments, the tissue sample may be mechanically disaggregated. In embodiments incorporating mechanical disaggregation, the tissue sample may be mechanically disaggregated by scraping with a scalpel or other scraping instrument until the dermis has disintegrated or nearly disintegrated. Other means of mechanical disaggregation include macerating, slicing, cutting, crushing, grating, pulverizing, or other ways of mechanical disaggregation known in the art.

The disaggregated tissue sample and cells may then be further rinsed and suspended in an effective amount of buffer, which in this instance may comprise at least an amount of buffer sufficient to form a cell suspension. The cell suspension may include a population of viable cells (e.g., living cells) and non-viable cells (e.g., non-living cells).

The cell suspension may then be allowed to rest for a period of time. In preferred embodiments, the cell suspension is allowed to rest for about 5 minutes to about 5 hours. In some variations, the cell suspension is allowed to rest for about 10 minutes to about 2 hours. In a preferred form, the cell suspension is allowed to rest for at least 30 minutes. In other embodiments, the cell suspension is allowed to rest for 5 hours to 24 hours. In some variations, the cell suspension is allowed to rest for more than 24 hours. During the disaggregation step and also during this resting time, the cells within the suspension may release tissue regeneration factors.

Tissue regeneration factors may comprise growth factors, cytokines, proteases, and stress signals involved in wound healing. These proteins may provide one or more of the following properties: they may be broadly mitogenic, immunomodulatory, pro-inflammatory, anti-inflammatory proteins, may comprise anti-microbial, damage-associated molecular patterns (DAMPs), or may comprise free nucleic acids, lipids, extracellular vesicles, exosomes, micro-vesicles, and apoptotic bodies. In some embodiments, the exosomes may be 40-100 nm, the micro-vesicles may be 100-1000 nm, and the apoptotic bodies may be more than 1000 nm.

The tissue regeneration factors of the bioactive suspension may include any and all secretome elements produced or released by the cell suspension cells, including but not limited to heat shock protein, such as for example HSP90, epidermal growth factor (EGF), brain-derived neurotrophic factor (BDNF), interleukin-33 (IL-33), macrophage colony-stimulating factor (MCSF), stem cell factor (SCF), growth hormone (GH), insulin-like growth factor binding protein 1 (IGFBP-1), heparin-binding epidermal growth factor-like growth factor (HB-EGF), amphiregulin (AR), interleukin 1 alpha (IL-1α), vascular endothelial growth factor (VEGF), transforming growth factor alpha (TGF-α), vascular endothelial growth factor receptor 2 (VEGFR2), tumor necrosis factor receptor 2 (TNF R2), basic fibroblast growth factor (bFGF), interleukin-1 receptor antagonist (IL-1ra), tissue inhibitor matrix metalloproteinase 1 (TIMP-1), stem cell factor receptor (SCF R), fibroblast growth factor 4 (FGF-4), tumor necrosis factor receptor 1 (TNF R1), intercellular adhesion molecule 1 (ICAM-1), cathelicidin peptide (LL-37), macrophage colony-stimulating factor receptor (MCSF R), hepatocyte growth factor (HGF), epidermal growth factor receptor (EGF R), antithrombin III (ATIII), tissue inhibitor matrix metalloproteinase 2 (TIMP-2), and high mobility group box protein 1 (HMGB1). It is expressly contemplated that tissue regeneration factors may comprise any molecule found in the secretome of any tissue cell.

In some preferred embodiments, the tissue regeneration factors of the present invention may comprise at least epidermal growth factor (EGF), stem cell factor (SCF), heparin-binding epidermal growth factor-like growth factor (HB-EGF), amphiregulin (AR), vascular endothelial growth factor (VEGF), transforming growth factor alpha (TGF-α), vascular endothelial growth factor receptor 2 (VEGFR2), fibroblast growth factor 4 (FGF-4), and hepatocyte growth factor (HGF). In some embodiments, the bioactive suspension may comprise these tissue regeneration factors plus one or more of the additional tissue regeneration factors named in the preceding paragraph. In some embodiments, the bioactive suspension may comprise these tissue regeneration factors plus high mobility group box protein 1 (HMGB1). In some embodiments, high mobility group box protein 1 (HMGB1) is present at least at 500,000 pg/ml.

Following the rest period, the cells may be separated out from the cell suspension. In preferred embodiments, one or more centrifuges disclosed herein is used to perform a cell separation procedure, such as but not limited to pelleting out the cells. In embodiments, the cell separation procedure may be accomplished via a differential pelleting centrifuge an analytical ultracentrifuge (AUC), a preparative ultracentrifuge, a density gradient centrifuge, or a different type of centrifuge capable of separating cells from solution, to separate out the cells. The centrifuge may, in some embodiments, be configured to remove cells and large non-cellular debris (>100 microns) from the cell suspension. In other embodiments, a passive filter may be used to separate the cells. In some embodiments, isolation of cells using magnetic beads or fluorescent activated cell sorting may be used to separate out cells.

For ease of description, the term “centrifuge” herein encompasses a passive filter. Similarly, the term “centrifuged” herein encompasses passing the cell suspension through a filter. These terms also encompass a combination of filter and centrifuge, or filtering and centrifuging, respectively.

Any suitable centrifuge, whether a passive filter or a centrifuge filter, capable of separating excessively large cellular aggregates from the suspension may be used to filter the cell suspension. In some variations, the centrifuge or filter size may be between 50 μm and 200 μm, or between 75 μm and 150 μm, with 100 μm being a preferred example. Those of skill in the art will recognize that any device or machine capable of performing centrifuge filtration according to the description herein may comprise the centrifuge and/or centrifuge filter.

In some embodiments, the supernate remaining after the cells have been separated out constitutes an embodiment of the bioactive suspension. Being decellularized, the bioactive suspension is useful as an allogeneic therapeutic, or in combination with treatment cells and/or added exogenous agents such as additional tissue regeneration factors or other elements known to be therapeutic for tissue regeneration.

Optionally, at any point after centrifuging, the bioactive suspension may be diluted. In some embodiments, dilution may comprise adding one or more quantities of buffer to the bioactive suspension.

Those of skill in the art will note that the step of separating out the cells and retaining the supernate as the bioactive suspension for use as a tissue regeneration therapeutic, particularly in the art of skin restoration, runs counter to the expectation of a person skilled in the art, who is likely to know and prefer the use of both cells and supernate in a single solution to treat a patient, or who might know to culture the cells, or to use the cells alone, rather than to discard the cellular component and retain the supernate. The successful harvesting, preparation, and therapeutic use of the bioactive suspension alone is therefore surprising, unexpected, and a significant step forward in the art. The methods and compositions disclosed herein that provide for a cell free bioactive suspension run counter to such conventional wisdom. The experimental data tending to show the cell free bioactive suspension as having effectiveness are surprising.

Optionally, prior to use, the bioactive suspension may be frozen at about −20 C to about −80 C. In other variations, the bioactive suspension may be stored in a frozen state at temperatures below −80 C. In some variations, the bioactive suspension may be frozen and stored from about −80 C to about −196 C, such as by storage using liquid nitrogen. If frozen, the bioactive suspension may be thawed and used as soon as practicable.

In some variations, the initial cell suspension may be prepared at least partially through any of the methods described in one or more of U.S. Pat. Nos. 9,029,140, 10,626,358, or U.S. patent application Ser. No. 16/935,977, each of which is incorporated herein its entirety herein. In some variations the initial cell suspension may be prepared at least partially through one or more automated methods, such as with a system similar to that described in U.S. Pat. No. 10,626,358 including a pestle or other suitable tissue disaggregating member with a surface suitable for performing physical tissue disruption e.g., grinding, scraping, shaving, etc.

The bioactive suspension formulated for use as a standalone therapeutic may be applied to a treatment area on a patient. It is contemplated that in such an administration, the bioactive suspension may enhance the patient's natural tissue regeneration.

The Bioactive Suspension in Combination with Treatment Cells

Optionally, a method of preparing the bioactive suspension for use as a cell therapy delivery agent may further comprise the step of adding one or more populations of treatment cells to the bioactive suspension. In some embodiments, the treatment cells may comprise cells obtained from the freshly disaggregated tissue used to obtain the bioactive suspension, cultured cells, gene-edited cells, induced pluripotent stem cells (iPSCs), forward-differentiated cells, or another type of cell. It is contemplated that any method of inducing pluripotency is compatible with the present disclosure, such as but not limited to a method that causes cells to express Yamanaka factors. In some embodiments, the treatment cells may comprise tissue cells from the freshly disaggregated tissue, cultured cells, or gene-edited cells. In some embodiments, the treatment cells may comprise forward-differentiated cells that express telomerase, or which have been genetically corrected to correct for a skin disease.

By way of nonlimiting example, in some embodiments, a tissue sample from a patient having a genetic disorder may be taken. Then, the tissue sample may be processed and a bioactive suspension prepared according to one or more methods described herein that utilizes a passive filter and collects viable cells. The collected cells may then be induced into a pluripotent state (reverse differentiated) by methods known in the art to yield induced pluripotent stem cells (iPSCs). The iPSC tissue cells may then be genetically corrected. The genetically corrected cells may then be forward differentiated into skin cells, these treatment cells may be combined with the bioactive composition, and the resulting bioactive suspension applied to a treatment site. In some embodiments, genetically corrected treatment cells and wild type treatment cells may comprise treatment cells and may be combined with the bioactive suspension, then applied to the treatment site.

The treatment cells may additionally or optionally comprise autologous cells, allogeneic cells, gene-edited autologous cells, gene-edited allogeneic cells, xenogeneic cells, gene-edited xenogeneic cells, or any combination of such cells. In some preferred embodiments, the treatment cells may comprise iPSCs.

In some preferred embodiments, the treatment cells may comprise gene-corrected induced iPSCs. In some preferred embodiments, the treatment cells may comprise gene-edited epidermolysis bullosa induced pluripotent stem cells (EB iPSC)-derived cells, wherein the defective epidermolysis bullosa related gene(s) have been corrected according to methods known in the art.

For example, in a preferred variation, a patient with a debilitating skin disorder such as epidermolysis bullosa may receive a treatment wherein gene-edited epidermolysis bullosa cells are combined with the bioactive suspension and administered to one or more portions of an epidermolysis bullosa patient's skin or wound bed. In an alternative embodiment, an epidermolysis patient may receive a treatment wherein gene-edited epidermolysis bullosa cells and wild-type healthy (i.e. not affected by epidermolysis bullosa) skin cells are combined with the bioactive suspension and administered to one or more portions of an epidermolysis bullosa patient's skin or wound bed.

In some embodiments, the treatment cells may comprise gene-edited cells that have been edited to correct for one or more defects known to cause or partially cause one or more diseases, such as but not limited to Autosomal Recessive Congenital Ichthyosis, Harlequin ichthyosis, Netherton syndrome, Hailey-Hailey disease, and Darier disease.

In some embodiments, the treatment cells may comprise cells that have been contacted with contact non-integrating reprogramming factors, such as but not limited to mRNA encoding nuclear-acting polypeptides. In some embodiments, the mRNA encoding nuclear-acting polypeptides may alter transcription and induce reprogramming in targeted cells. Any use of mRNA encoding nuclear-acting polypeptides to alter transcription and induce reprogramming in targeted cells is contemplated. One non-limiting example of the foregoing may comprise one or more reprogramming factors comprising mRNA encoding telomerase, and/or its RNA subunit TERC, and/or telomere associated proteins (e.g. the shelterin proteins). In some embodiments, said contact may comprise transfection with hTERT or p-hTERT modified messenger RNAs (mmRNAs).

In embodiments that include treatment cells, the selected treatment may comprise seeding a treatment area with a bioactive suspension with treatment cells, which may result in enhanced tissue regeneration of the treatment cells on the treatment area. In some embodiments, gene-edited cells may be cultured with the bioactive suspension prior to application to enhance therapeutic potential.

It is contemplated that any type of cell-based therapy is amenable for combination with the bioactive suspension. Due at least in part to the tissue regeneration factors of the bioactive suspension, the treatment cells of the cell-based therapy, when administered in combination with the bioactive suspension, may adhere, grow, or persist in an improved manner as compared with the administration of treatment cells without the presence of the bioactive suspension.

The Bioactive Suspension in Combination with Added Efficacious Elements

Optionally, after obtaining the bioactive suspension, in some embodiments, the bioactive suspension may be supplemented by the addition of one or more added efficacious elements, such as for example hyaluronic acid, heat shock protein(s), platelet-enriched plasma, adipose stem cells, mesenchymal stem cells, or one or more tissue regeneration factors supplied from a source other than the tissue sample used to make the bioactive suspension.

The at least one efficacious element may alternatively or additionally comprise at least one phytoconstituent or other plant-based biochemical. In some embodiments, the at least one phytoconstituent may be obtained via decoction, infusion, maceration, percolation, solvent extraction, steam distillation, Soxhlet extraction, enzymatic digestion, or any other method known in the relevant art.

Illustrative phytoconstituents may comprise, by way of nonlimiting examples, alkaloids, steroids, sterols, polyphenols, triterpenes, glycosides, carbohydrates, proteins, amino acids, flavonoids, saponins, phenolic compounds, and tannins.

In some embodiments, the at least one phytoconstituent may comprise a phytoconstituent derived from one or more of Celosia argentea, Buddleja globosa, Onosma argentatum, Scrophularia nodosa, Chromolaena odorata, Combretum smeathmanni, Phyllanthus muellerianus, Pycnanthus angolensis, Aloe vera, Alternanthera brasiliana, Dalbergia odorifera, Epimedium sagittatum, Trichosanthes kirilowii, Allamanda cathartica, Anogeissus latifolia, Carallia brachiate, Cynodon dactylon, Allium cepa, Areca catechu, Berberis lyceum, Carapa guainensis, Carica papaya, Acacia nilotica, Aspilia Africana, Emblica officinalis, Memecylon umbellatum, Rubia cordifolia, Achillea millefolium, Centella asiatica, Cocos nucifera, Crocus sativus, Cannabis sativa, Cannabis indica, Cannabis ruderalis, Laurus nobilis, Kaempferia galangal, Cecropia pelfata, Lawsonia inormis, Radix paeoniae, Lycopodium serratum, Jasminum grandiflorum, Bambusa vulgaris, Utleria solicifolia curcas, Clerodendrum infortunatu, Centella asiatica, Comphora officinarum, Shorea robusta, Apis mellifera, Sesamum indicum, Azardica indica, Vitex nigundo, Emblica officinalis (in some embodiments, the bark) Gaertn, Tridox procumbens, Arctium lappa, Astragalus propinquus, Rehmannia glutinosa, Ampelopsis japonica, Andrographis paniculata, Angelica sinensis, Blumea balsamifera, Boswellia sacra, Agrimonia eupatoria, Nelumbo nucifera, Boswellia sacra, pollen from Typha angustifoliae, Caesalpinia sappan, Calendula officinalis, Camellia sinensis, Carthamus tinctorius, Celosia argentea, Centella asiatica, Cinnamomum cassia, Commiphora myrrha, Curcuma longa, Daphne genkwa, Entada phaseoloides, Hibiscus rosa-sinensis, Ganoderma lucidum, Ligusticum striatum, Lonicera japonica, Paeonia suffruticosa, Panax ginseng, Panax notoginseng, Polygonum cuspidatum, Lithospermum erythrorhizon, Rheum officinale, Rhodiola imbricata, Salvia miltiorrhiza, Sanguisorba officinalis, Sophora flavescens, Stemona tuberosa, Wedelia trilobata, Zanthoxylum bungeanum, common lavender, chamomile, echinacea, rosemary, hemp, turmeric, orange flower, jasmine, avocado, and flaxseed, as well as any plant known in the art to provide one or more compounds that accelerate the healing process. In some embodiments, the at least one phytoconstituent may comprise a compound derived from an essential oil selected from the group comprising African lemon bush (Lippia javanica) oil, anise oil, bay oil, bergamot oil, boronia oil, cannabis oil, canola oil, carrot oil, cassia oil, catnip oil, cedarwood oil, chamomile oil, cinnamon oil, citronella oil, clary sage oil, clove oil, cypress oil, eucalyptus oil, galbanum oil, garlic oil, ginger oil, geranium oil, grapefruit oil, hazelnut oil, hemp oil, jasmine oil, jojoba oil, lavender oil, lavandin oil, lemon oil, lime oil, mandarin oil, nutmeg oil, orange oil, palma rosa oil, patchouli oil, Peru balsams, peppermint oil, rosemary oil, rosewood oil, sage oil, sandalwood oil, spear mint oil, star anise oil, tea tree oil, tangerine oil, thyme oil, tolu, verbena oil, white clover oil, ylang ylang oil, and combinations thereof.

In some embodiments, the at least one efficacious element may comprise a trace element such as, by way of nonlimiting example, iron, manganese, zinc, copper, magnesium, nickel, boron, or molybdenum, or one or more vitamins selected from the group comprising vitamin A, vitamin B6, vitamin B12, vitamin C, vitamin D, vitamin E, folic acid, K, niacin, and the like, or calcium, phosphorus, potassium, sodium, chlorine, sulfur, fluoride, vanadium, nitrogen, chromium, iodine, and selenium.

In some embodiments, the at least one efficacious element may comprise a Janus kinase inhibitor (“JAK inhibitor”). Nonlimiting examples of the at least one JAK inhibitor comprise Abrocitinib, Delgocitinib, Fedratinib, Ruxolitinib, Upadacitinib, Tofacitinib, Baricitinib, Peficitinib, Filgotinib, Fedratinib, Oclacitinib, Cerdulatinib, Itacitinib, Gandotinib, Lestaurtinib, Momelotinib, Pacritinib, Deucravacitinib, Cucurbitacin I, CHZ868, SHR0302, and BMS-986165. In some embodiments, the JAK inhibitor may be an extract of one or more plants, such as but not limited to blackberry, boysenberry, feijoa, pomegranate, rosehip and strawberry, or any plant containing one or more ellagitannins. In some embodiments, the plant source of JAK inhibitors is from the Rosales or Myrtales plant orders. Nonlimiting examples of ellagitannins include Castalagin, Castalin, Casuarictin, Grandinin, Roburin A, Tellimagrandin II, Terflavin B, Vescalagin, Pomegranate ellagitannins, Punicalagin, and Punicalin.

In some embodiments, the at least one efficacious element may comprise an enzyme inhibitor. Nonlimiting examples include a protease inhibitor, carbohydrase inhibitors, lipase inhibitors, or any oxidoreductase inhibitor, transferase inhibitor, hydrolase inhibitor, lyases inhibitor, isomerase inhibitor, ligase inhibitor, matrix metalloproteinases, and translocase inhibitor.

In some embodiments, the at least one efficacious element may comprise an antibacterial agent such as but not limited to ciprofloxacin, amoxicillin-clavulanate, cephalexin, clindamycin, dicloxacillin, doxycycline, trimethoprim-sulfamethoxazole, honey, acetic acid, iodine, povidone iodine, cadexomer iodine, silver sulphadiazine (SSD), bacitracin, mafenide, mupirocin, neomycin, curcumin, and any other antibacterial agent known to be compatible with the present disclosure.

### The Bioactive Suspension as Reagent for Enhanced Cell Growth in Culture

In some embodiments, the bioactive suspension may comprise a reagent for enhanced cell growth in culture. In such embodiments, certain steps in the method may be modified to optimize the bioactive suspension's properties, including but not limited to the selection of tissue for harvesting, what type of treatment cells are added, and whether to add the treatment cell(s) at the step of resting, after separation, or just prior to administration. Moreover, in some embodiments the bioactive suspension may further comprise added efficacious elements, such as nutrients or pH-adjusting elements, that may enhance the viability of certain cells in culture, such as but not limited to cells that are known to be difficult to culture.

### Methods of Preparing a Synthetic Bioactive Suspension

In one or more alternative embodiments, the bioactive suspension may be formulated apart from the use of freshly disaggregated tissue to form a synthetic bioactive suspension. In some embodiments, a synthetic bioactive suspension may comprise an effective amount of buffer solution to which one or more of tissue regeneration factors, efficacious elements, or treatment cells may be added. In some embodiments, a combination of two or more tissue regeneration factors, efficacious elements, or treatment cells may be added to an effective amount of buffer to comprise the synthetic bioactive suspension.

In some embodiments of a synthetic bioactive suspension, it is contemplated that such tissue regeneration factors, efficacious elements, and/or treatment cells may be separately produced according to one or more methods disclosed herein, or another method known in the art, and then added to an effective amount of buffer. In some embodiments of a synthetic bioactive suspension, it is contemplated that such tissue regeneration factors, efficacious elements, and/or treatment cells may be purchased from a commercial supplier and then added to the effective amount of buffer. It is contemplated that the step of formulating the tissue regeneration factors, efficacious elements, and/or treatment cells with the buffer to produce a synthetic bioactive suspension may be performed according to known laboratory techniques.

The synthetic bioactive suspension may, in some embodiments, be combined with one or more scaffolding elements as well to form a synthetic bioactive suspension product. All examples provided herein of skin grafts and scaffolding elements are contemplated to be combined with the synthetic bioactive suspension, including but not limited to meshed split-thickness skin grafts, temporary dressings, permanent dressings, liquids, spray formulations, spray bandages, gels, bioprinted gels, and any other scaffolding element disclosed herein.

### Method of Preparing a Bioactive Suspension Combination Product

In some embodiments, one or more bioactive suspensions made according to any method described herein may be combined with an additional therapeutic element, such as by way of nonlimiting illustration, a matrix, an autologous skin graft, or a cream, to comprise a bioactive suspension combination product. In some embodiments, a bioactive suspension combination product may serve dual purposes as both an agent of wound closure and of tissue regeneration. It is contemplated therefore that one or more bioactive suspension combination products may comprise or function as a bandage, temporary dressing, permanent dressing, bandage healing aid, skin healing cream, or any other topical use germane to the present disclosure.

In some embodiments, the scaffolding element may comprise a skin graft, an extracellular matrix, or other matrix material. Examples of scaffolding elements comprising skin grafts include, but are not limited to, one or more of human skin, a meshed split-thickness skin graft, a split-thickness skin graft, a split-thickness human skin sample, a full thickness human skin sample, human skin epidermis, human skin dermis, human skin epidermal-dermal junction tissue, human mesenchymal stem cells, human adipose tissue, non-human epidermis, non-human dermis, non-human epidermal-dermal junction tissue, non-human mesenchymal stem cells, and non-human adipose tissue.

In various embodiments, the scaffolding element may be synthetic or biological, such as collagen, alginate, alginate beads, agarose, fibrin, fibrin glue, fibrinogen, blood plasma fibrin beads, whole plasma or components thereof, laminins, fibronectins, proteoglycans, HSP, chitosan, heparin, and/or other synthetic polymer or polymer scaffolds and solid support materials, such as wound dressings, that could hold or adhere to cells. In some embodiments, the scaffolding element may comprise a tissue or organ regeneration system (in vivo or in vitro).

In some embodiments, the scaffolding element may comprise polymeric materials, such as but not limited to synthetic polymer films, foam dressings, hydrocolloids, alginate dressings, and hydrogels, polymeric foam, polymeric hydrogels, polymeric alginates, polymeric hydrocolloides, passive synthetic polymer dressings, interactive synthetic polymer dressings, polymer films, polyurethane (PU), semi-occlusive film, occlusive film, polyurethane foam film dressing, hydrophilic polymeric foam dressing, polysaccharides, agar, alginate, alginate dressings, carrageenans, pectin, gelatin, carboxymethylcellulose, crosslinked polymers (hydrophilic) such as polyvinylpyrrolidone, polyacrylamide, and polyethylene oxide, poly(lactide-co-glycolide) (PLGA), polyethylene glycol (PEG), PEG blended with chitosan and PLGA, polycaprolactone (PCL), electrospun PCL fibers, fucoidan, sulphated polysaccharides, silk sericin, Acetobacter xylinum utilizes carbon from nutrition media and form beta 1-4 glucose in the form of linear chains, keratin, hyaluronic acid dressings, homoglycans such as starch, cellulose, and dextran, pallulan, yeast, grains and fungi yield beta glucans which can form double and triple helix resistant gel, bovine serum albumin, bi-layered bioengineered skin substitute (BBSS) acellular (collagen) matrixes from porcine small intestines, cultured allograft and autograft epidermal sheets, allografts from cadaveric skins, tissue-engineered skin substitutes, collagen-glucosamine skin scaffolds, sericin (silkworm) matrices, dermal substitutes, animal derived acellular xenografts, shark derived matrix of bovine collagenase chondroitin-6-sulfate and disposable silicone sheet, nylon mesh crosslinked with the porcine collagen, bi-layered substitute with a removable semi-permeable silicone layer, acellular dermal allografts, cadaveric-derived acellular dermal allografts, temporary bioengineered skin substitute, neonatal fibroblasts cultured on nylon fiber that are embedded into a silastic layer for 4-6 weeks and form dense cellular tissue, absorbable polyglactin scaffold colonized with allogenic neonatal fibroblasts, composite grafts, collagen scaffold, cultured fibroblasts and a layer of stratified cultured human keratinocytes, cultured neonatal keratinocytes and bovine collagen, composite skin graft, skin equivalent, organo-typical skin substitute, compositions of both living dermis and epidermis, composite bi-layer products, bi-layer bioengineered skin, fibroblast seeded scaffolds, adipose-derived human lipoaspirate from embryonic mesenchyme, non-contact radiant heat bandages, micro- and nanoparticulate systems, nanogels, novel nanofibrous chitosan-Fb scaffold, electrospun nanofibrous hybrid wound-dressing from PCL/collagen, electrospun nanofibers enriched with quercetin and ciprofloxacin hydrochloride, PCL/gelatin hybrid composite mats, microfibrous constructs with Ag nanoparticles, fibroblast seeded PU/SF scaffolds, biocompatible natural carbohydrate polymeric dressings including chitosan, microbe-derived polysaccharides such as microbial cellulose, and any other polymeric biomaterial known to be compatible with the present disclosure.

In some embodiments, the scaffolding element may comprise a fibrin glue, also known as a fibrin sealant or fibrin tissue adhesive, any other hemostatic, tissue sealant, and tissue adhesive; primary polymer-based dressing, or a primary wound spray dressing such as but not limited to wound spray, liquid bandage, first aid spray, antibacterial polymersome-based wound dressing spray; oil based bactericides/virucides spray dressings; and any other spray or liquid-applied bandage substitute.

In some embodiments, the scaffolding element may comprise bioprinted tissue, such as by way of illustration and not limitation, bioprinted skin tissue, a bioprinted gel configured for application to a skin wound. In some embodiments, the scaffolding may comprise a different type of bioprinted tissue, such as muscle, bone, nerve, or connective tissue.

A bioactive suspension combination product may in some embodiments comprise a bioactive suspension made according to one or more methods disclosed herein and in combination with one or more scaffolding elements, such as but not limited to autologous split-thickness skin grafts, or synthetic or biologic scaffolds, to treat larger and deeper wounds. It may be used as a single therapeutic approach or following a dosing regimen.

In some embodiments, the bioactive suspension combination product may be formulated as the bioactive suspension made according to one or more methods disclosed herein and combined with a topical scaffolding element, such as a cream, lotion, or paste, which may be manually or mechanically applied to a treatment area. In such embodiments, the bioactive suspension may further comprise one or more thickening or conditioning agents configured to deliver the bioactive suspension as a cream, lotion, or paste.

### Bioactive Suspension Compositions

Bioactive suspension compositions made as product by process of any method described herein are expressly contemplated, such as but not limited to a cell-free and added element-free bioactive suspension composition made according to one or more methods disclosed herein, a bioactive suspension composition further comprising treatment cells made according to one or more methods disclosed herein, a bioactive suspension composition further comprising added efficacious elements as disclosed herein, a bioactive suspension combination product made according to one or more methods disclosed herein, and a synthetic bioactive composition made according to one or more methods disclosed herein.

For clarity, a bioactive suspension composition may comprise the product of a process comprising (1) obtaining a tissue sample, (2) subjecting the tissue sample to disaggregation, [which in some embodiments may comprise (a) enzymatic disaggregation followed by rinsing residual enzyme; (b) mechanical disaggregation; or (c) enzymatic disaggregation followed by mechanical disaggregation], (3) adding additional buffer to obtain a cell suspension, (4) allowing the cell suspension to rest for an effective amount of time, (5) separating the cells of the cell suspension out of the suspension to obtain a bioactive suspension, (6) optionally, freezing and then thawing the bioactive suspension, (7) optionally, adding one or more populations of treatment cells to the bioactive suspension to obtain a bioactive suspension configured for use as a cell therapy delivery agent, (8) optionally, adding one or more tissue regeneration factors to the bioactive suspension, and (9) optionally, adding one or more efficacious elements to the bioactive suspension.

### Methods of Administering the Bioactive Suspension

In some variations, the bioactive suspension may be applied directly to a recipient region. For example, if the recipient region comprises an acute wound, a chronic wound, a soft tissue injury, a burn, skin that has been ablated in preparation for a treatment, such as but not limited to a vitiligo treatment, or any partial-thickness skin injury, then the bioactive suspension may be applied directly to the recipient region.

In variations of the bioactive suspension that include treatment cells, the bioactive suspension with treatment cells may be applied such that at least a threshold density of viable cells (number of viable cells per unit surface area) is administered across the surface area of the recipient region. This threshold of viable cell distribution may help ensure sufficiently dense coverage of viable cells across the recipient region for the cell suspension to utilize the therapeutic potential of the cell suspension. It is to be understood that the threshold of viable cell distribution will vary considerably, but experimental results show that a surprisingly low number of viable cells may be effectively administered in combination with the bioactive suspension.

The bioactive suspension may be applied using one or more suitable application techniques. In some variations, at least a portion of the bioactive suspension may be applied via spray application technique. For example, a spray applicator such as a syringe, a spray nozzle, a syringe attached to a spray nozzle, a powered spray device such as a “spray gun” or other such delivery devices may be used to apply the bioactive suspension. The spray applicator may be held such that the nozzle may face the wound. The spray applicator may be held at approximately 10 cm from the most elevated point of the recipient region such that the first drop of bioactive suspension falls onto the recipient region. The bioactive suspension may be sprayed from the most elevated point of the recipient region such the any run-offs may cover more dependent areas of the recipient region. A fine mist of bioactive suspension may be delivered to the recipient region. In some variations, if the recipient region is large, the spray applicator may be moved in continuous motion from one part of the recipient region to another part of the recipient region as the bioactive suspension is being sprayed.

In some variations (e.g., requiring an application of lesser than 2 ml of the bioactive suspension), at least a portion of the bioactive suspension may be applied via drip application technique. In such variations, a syringe may be held adjacent to or a few millimeters away from an elevated point of the recipient region such that the bioactive suspension is carefully dripped onto the recipient region. Any run-off may cover more dependent areas of the recipient region.

Moreover, in some embodiments, the bioactive suspension may be formulated as an injectable solution. In such embodiments, the bioactive suspension may comprise more or less water or other liquid solution so as to ensure that the viscosity of the bioactive solution is appropriate for administration by injection.

In another variation, the bioactive suspension may be prepared by combining certain tissue regeneration factors with cell culture media, Lactated Ringers solution, or other suitable media or solution.

In some embodiments, the bioactive suspension may be combined with one or more scaffolding elements prior to the application of this combination to a treatment site.

### Preliminary Characterizations of the Bioactive Suspension

Preliminary characterization work has been conducted demonstrating the release of broad mitogenic, immunomodulatory, pro-inflammatory, anti-inflammatory, anti-microbial, and danger associated molecular patterns important in the wound healing cascade (Table 1).

TABLE 1
Initial screen of wound healing factors present in the bioactive
suspension (prepared using RECELL ® Buffer).

| <20 | 20-500 | 500-1000 | 2500-6000 | >500,000 |
|---|---|---|---|---|
| pg/ml | pg/ml | pg/ml | pg/ml | pg/ml |
| EGF | IL-1a | ICAM-1 | ATIII | HMGB-1 |
| BDNF | VEGF | LL-37 | TIMP-2 | |
| IL-33 | TGFa | MCSF R | | |
| MCSF | VEGF R2 | HGF | | |
| SCF | TNF RII | EGFR | | |
| GH | bFGF | | | |
| IGFBP-1 | IL-1ra | | | |
| HB-EGF | TIMP-1 | | | |
| AR | SCF R | | | |
| | FGF4 | | | |
| | TNF R1 | | | |

For clarity regarding Table 1, in at least one experiment, epidermal growth factor (EGF), brain-derived neurotrophic factor (BDNF), interleukin-33 (IL-33), macrophage colony-stimulating factor (MCSF), stem cell factor (SCF), growth hormone (GH), insulin-like growth factor binding protein 1 (IGFBP-1), heparin-binding epidermal growth factor-like growth factor (HB-EGF), and amphiregulin (AR) were found to be present at <20 pg/ml of the bioactive suspension.

For clarity regarding Table 1, interleukin 1 alpha (IL-1α), vascular endothelial growth factor (VEGF), transforming growth factor alpha (TGF-α), vascular endothelial growth factor receptor 2 (VEGFR2), tumor necrosis factor receptor 2 (TNF RII), basic fibroblast growth factor (bFGF), interleukin-1 receptor antagonist (IL-1ra), tissue inhibitor matrix metalloproteinase 1 (TIMP1), stem cell factor receptor (SCF R), fibroblast growth factor 4 (FGF4), and tumor necrosis factor receptor 1 (TNFR1) were found to be present at 20-500 pg/ml of the bioactive suspension.

For clarity regarding Table 1, intercellular adhesion molecule 1 (ICAM-1), cathelicidin peptide LL-37, macrophage colony-stimulating factor receptor (MCSF R), hepatocyte growth factor (HGF), and epidermal growth factor receptor (EGFR) were found to be present at 500-1000 pg/ml of the bioactive suspension.

For clarity regarding Table 1, antithrombin III (ATIII), and tissue inhibitor matrix metalloproteinase 2 (TIMP-2) were found to be present at 2500-6000 pg/ml of the bioactive suspension.

For clarity regarding Table 1, high mobility group box protein 1 (HMGB1) was found to be present at >500,000 pg/ml of the bioactive suspension.

Using an in vitro model system, it has been shown discovered that the delivery of isolated keratinocytes resuspended in the bioactive suspension supports a more rapid formation of an epidermis on a dermal substitute, compared to traditional cell seeding techniques (FIG. 3). Furthermore, it was demonstrated that similar results were seen using half of the cell number typically seeded (FIG. 4).

### Experimental Design

In Vitro Evaluation of De Novo Epidermal Development Using RES® Regenerative Epidermal (RES) Bioactive Suspension

Bioactive suspension was prepared from freshly discarded tissue from surgically excised human skin using the RECELL® System. The RECELL® Autologous Skin Cell Harvesting Device was used to process the bioactive suspension following the Instructions for Use. 30 minutes following preparation of RES, the bioactive suspension was prepared by centrifugation at 500×g for 5 min at room temperature. Following separation, the bioactive suspension was removed and stored in 50 ml conical tube. The bioactive suspension was used to resuspend primary cultured human keratinocytes on constructs consisting of electrospun type I collagen and cultured primary human fibroblasts (dermal equivalent).

At days 7, 10, and 14 skin samples were collected, embedded in OCT and cooled. Cooled blocks were cryosectioned at a thickness of 7 μm and stained with hematoxylin and eosin. Sections were imaged.

### Animal Study Methods

A porcine study was conducted to examine the impact of a bioactive suspension on re-epithelialization and on the expression of various proteins in the wound bed during the acute healing phase.

Study design: Female Yorkshire pigs were acquired and acclimated in the animal facility prior to start of the study. Full-thickness excisional wounds measuring 4 cm×4 cm were prepared on the backs of 6 animals. Two treatment groups were evaluated at two timepoints (Day 3 and Day 6). Three pigs were sacrificed at Day 3 and 3 pigs were sacrificed at Day 6. The treatment groups were as follows:

Autologous split-thickness skin graft (STSG) (3:1 mesh) Day 3 (n=6 wounds) and Day 6 (n=6 wounds)

STSG meshed at 3:1 ratio (mSTSG)+a bioactive suspension Day 3 (n=6 wounds) and Day 6 (n=6 wounds).

Full-thickness Excision: Full-thickness defects were created (surgical excision) containing the entire epidermis and dermis as well as the fat layer above the fascia.

Skin Harvesting for mSTSGs and generation of bioactive suspension: Skin grafting was performed following full-thickness excision. The area of skin required for mSTSGs and bioactive suspension generation was harvested using a dermatome. Autografts were collected at a depth 0.010-0.012″ for the mSTSGs and at 0.006-0.008″ for the bioactive suspension. Skin allocated to meshing was prepared using a meshing ratio of 3:1 using a Zimmer skin graft mesher. Skin was used at the time of harvest (i.e., on the day of surgery and not at a later time point).

Application of 3:1 Meshed STSG: All wounds received 3:1 meshed autograft. Each wound had a fully expanded 3:1 mSTSG placed in the wound and sutured or stapled to secure.

Preparation of bioactive suspension: the RECELL® System was used to derive a cell suspension at an expansion ratio of 1:5. After completion of RES preparation the resulting suspension was incubated at Room Temperature (RT) for 30 minutes prior to centrifugation at 600×g for 5 minutes at RT. The resulting bioactive suspension was collected by carefully pipetting the supernate as to not disturb the cell pellet and transferred to a new tube.

Bioactive Suspension Treatment: Following application of mSTSG, each wound received either no additional treatment, or the following treatment: Bioactive suspension prepared at a 1:5 expansion ratio was applied to the wound bed.

Dressings: The wounds were dressed with Telfa Clear followed by Xeroform, Absorbent dressings, KCI Vac Drape, Elastikon and a Pig Jacket.

Dressing Changes, Data Collection, and Biopsy Acquisition: Dressing changes, data collection, and punch biopsy acquisition were performed under sedation. Dressings were changed at 72 hrs (±1 day), unless an earlier timepoint was required based on clinical observations. On either Day 3 or Day 6 biopsies were harvested and placed in either 10% Formalin, OCT, or snap frozen for downstream analysis.

Trans-Epidermal Water Loss: For animals survived to day 6, TEWL measurements were performed on wound sites within each site, in addition to uninjured skin.

Histology and Pathology: Formalin fixed biopsies were processed, paraffin embedded and sectioned/mounted for downstream applications. Sections were stained with H&E to assess re-epithelialization and an anti-Ki67 antibody to assess cellular proliferation. Re-epithelialization Analysis: Wound re-epithelialization rate was assessed by measuring a) the interstice width at the level of epithelium and b) the length of the re-epithelized epithelia from the two interstice's edges. Percent re-epithelialization was calculated as the total length of epithelium normalized to the interstice width.

For each treatment group (mSTSG alone and mSTSG plus a bioactive suspension), the values of the two replicate wounds per pig were averaged, representing one re-epithelialization % per pig per group (Total=3 values per group/time point).

FIG. 6 depicts a graph showing experimental day 6 re-epithelialization results of wounds treated with a mSTSG and an embodiment of the bioactive suspension compared to wounds treated with the mSTSG alone. Wounds treated with a mSTSG and the addition of a bioactive suspension had greater average re-epithelialization 6 days after wounding/treatment compared to wounds treated with the mSTSG alone.

FIG. 7 depicts a graph showing Ki67 staining results of wounds treated with a mSTSG and an embodiment of the bioactive suspension compared to wounds treated with the mSTSG alone. The number of epidermal Ki67+ cells were counted per 40× Grid per μm². Total Ki67+ counts from two replicate wounds per pig were averaged, representing one Ki67+ count per pig per group (Total=3 values per group/time point). Wounds treated with a mSTSG and the addition of a bioactive suspension had more proliferative, Ki67 positive cells at day 3 than wounds treated only with a mSTSG.

FIG. 8 depicts a graph showing transepidermal water loss (TEWL) results of wounds treated with a mSTSG and an embodiment of the bioactive suspension compared to wounds treated with the mSTSG alone. On Day 6, wounds were measured for TEWL using the Tewameter® TM Nano probe (Courage+Khazaka electronic GmbH). Three repeated measurements were taken for each wound as well as for control uninjured tissue. Repeated measurements from the two replicate skin/wound per pig were averaged, representing one TEWL reading per pig per group (Total=3 values per group/location).

Wounds treated with a mSTSG and bioactive suspension had lower transepidermal water loss than wounds treated only with a mSTSG indicating a more robust epidermal barrier when a bioactive suspension was added.

FIG. 9A and FIG. 9B depict graphs showing protein concentrations for the bioactive suspension over time. Protein concentration measurement of the bioactive suspension was performed using BCA (bicinchoninic) protein assay, a colorimetric detection and quantitation method for measuring total protein in a solution. FIG. 9A shows a time-course analysis of the 1:80 expansion ratio preparation. No significant difference was seen in the protein concentration present in the bioactive suspension, and incubation of the bioactive suspension at 37 C did not have significant impact on the protein concentration. FIG. 9B shows that bioactive suspension made from a 1:5 expansion ratio is shown to have ˜10 times the total protein concentration as bioactive suspension obtained from a 1:80 concentration.

FIG. 10A, FIG. 10B, and FIG. 10C depict graphs showing expression levels of selected proteins involved in wound re-epithelialization in the wound bed of wounds treated with a mSTSG and an embodiment of the bioactive suspension compared to wounds treated with mSTSG alone. Wounds treated with a mSTSG and the addition of bioactive suspension had elevated levels of selected proteins at 3 days and significant elevated levels at 6 days compared to wounds treated with mSTSG alone. The legend in FIG. 10C applies to FIG. 10A and FIG. 10B, namely that circles indicate mSTSG and triangles indicate mSTSG+bioactive suspension in at least one embodiment,

Methods: Protein expression analysis: A large cross section of the wound bed was harvested at 3- or 6-days post initial treatment and total protein was extracted from the samples. Sample concentrations were determined using Bradford Assay. Quantibody® (an array-based multiplex ELISA) for Porcine (pig) Cytokine Array Kit was performed for quantitative measurement of selected protiens involved in the wound healing response. Normalized protein expression values (pg/ml) of selected cytokines in each group (mSTSG and mSTSG+Bioactive suspension) were calculated by averaging the values of each wound from each biological replicate (N=3/time point) in each group.

### Embodiments

A method of producing a bioactive suspension, comprising the steps of: obtaining a tissue sample; warming an enzyme solution to an effective temperature; contacting the tissue sample with at least one quantity of enzyme solution for at least one period of enzymatic disaggregation time; rinsing the tissue sample with a first quantity of buffer; mechanically disaggregating the tissue sample to produce a disaggregated tissue sample; adding a second quantity of buffer to the disaggregated tissue sample to create a cell suspension; allowing the cell suspension to rest of an effective amount of time; and separating the tissue cells from the cell suspension by at least one cell separation procedure to produce the bioactive suspension, wherein the bioactive suspension contains at least one tissue regeneration factor.

The method of any other embodiment, further comprising the step of adding at least one efficacious element.

The method of any other embodiment, further comprising the step of adding at least one population of treatment cells to the bioactive suspension.

The method of any other embodiment, further comprising the step of applying the bioactive suspension to at least one treatment site on a patient.

The method of any other embodiment further comprising, after the step of separating the tissue cells, the steps of freezing the bioactive suspension then thawing the bioactive suspension, and then applying the bioactive suspension to at least one treatment site.

The method of any other embodiment further comprising, after the step of separating the tissue cells, the steps of freezing the bioactive suspension; thawing the bioactive suspension; adding at least one population of treatment cells to the bioactive suspension; and applying the bioactive suspension to at least one treatment site.

The method of any other embodiment, wherein the step of freezing the bioactive suspension comprises freezing the bioactive suspension at −80 C or colder.

The method of any other embodiment further comprising, prior to applying the bioactive suspension, the step of adding the bioactive suspension to at least one scaffolding element.

The method of any other embodiment, wherein the at least one scaffolding element is selected from the group consisting of a gel matrix, a dressing, a bioprinted gel or a bioprinted tissue.

The method of any other embodiment, wherein the at least one scaffolding element is selected from the group consisting of human skin, a meshed split-thickness skin graft, a split-thickness skin graft, a split-thickness human skin sample, a full thickness human skin sample, human skin epidermis, human skin dermis, human skin epidermal-dermal junction tissue, human mesenchymal stem cells, human adipose tissue, non-human epidermis, non-human dermis, non-human epidermal-dermal junction tissue, non-human mesenchymal stem cells, and non-human adipose tissue.

The method of any other embodiment, wherein the at least one scaffolding element is selected from the group consisting of collagen, alginate, alginate beads, agarose, fibrin, fibrin glue, fibrinogen, blood plasma fibrin beads, whole plasma or components thereof, laminins, fibronectins, proteoglycans, HSP, chitosan, and heparin.

The method of any other embodiment, wherein the at least one scaffolding element is selected from the group consisting of synthetic polymer films, foam dressings, hydrocolloids, alginate dressings, and hydrogels, polymeric foam, polymeric hydrogels, polymeric alginates, polymeric hydrocolloides, passive synthetic polymer dressings, interactive synthetic polymer dressings, polymer films, polyurethane (PU), semi-occlusive film, occlusive film, polyurethane foam film dressing, hydrophilic polymeric foam dressing, polysaccharides, agar, alginate, alginate dressings, carrageenans, pectin, gelatin, carboxymethylcellulose, crosslinked polymers (hydrophilic) such as polyvinylpyrrolidone, polyacrylamide, and polyethylene oxide, poly(lactide-co-glycolide) (PLGA), polyethylene glycol (PEG), PEG blended with chitosan and PLGA, polycaprolactone (PCL), electrospun PCL fibers, fucoidan, sulphated polysaccharides, silk sericin, Acetobacter xylinum utilizes carbon from nutrition media and form beta 1-4 glucose in the form of linear chains, keratin, hyaluronic acid dressings, homoglycans such as starch, cellulose, and dextran, pallulan, yeast, grains and fungi yield beta glucans which can form double and triple helix resistant gel, bovine serum albumin, bi-layered bioengineered skin substitute (BBSS) acellular (collagen) matrixes from porcine small intestines, cultured allograft and autograft epidermal sheets, allografts from cadaveric skins, tissue-engineered skin substitutes, collagen-glucosamine skin scaffolds, sericin (silkworm) matrices, dermal substitutes, animal derived acellular xenografts, shark derived matrix of bovine collagenase chondroitin-6-sulfate and disposable silicone sheet, nylon mesh crosslinked with the porcine collagen, bi-layered substitute with a removable semi-permeable silicone layer, acellular dermal allografts, cadaveric-derived acellular dermal allografts, temporary bioengineered skin substitute, neonatal fibroblasts cultured on nylon fiber that are embedded into a silastic layer for 4-6 weeks and form dense cellular tissue, absorbable polyglactin scaffold colonized with allogenic neonatal fibroblasts, composite grafts, collagen scaffold, cultured fibroblasts and a layer of stratified cultured human keratinocytes, cultured neonatal keratinocytes and bovine collagen, composite skin graft, skin equivalent, organo-typical skin substitute, compositions of both living dermis and epidermis, composite bi-layer products, bi-layer bioengineered skin, fibroblast seeded scaffolds, adipose-derived human lipoaspirate from embryonic mesenchyme, non-contact radiant heat bandages, micro- and nanoparticulate systems, nanogels, novel nanofibrous chitosan-Fb scaffold, electrospun nanofibrous hybrid wound-dressing from PCL/collagen, electrospun nanofibers enriched with quercetin and ciprofloxacin hydrochloride, PCL/gelatin hybrid composite mats, microfibrous constructs with Ag nanoparticles, fibroblast seeded PU/SF scaffolds, biocompatible natural carbohydrate polymeric dressings including chitosan, and microbe-derived polysaccharides.

The method of any other embodiment, wherein the at least one scaffolding element is selected from the group consisting of fibrin sealant, fibrin tissue adhesive, hemostatic, tissue sealant, and tissue adhesive, primary polymer-based dressing, primary wound spray dressing, liquid bandage, first aid spray, antibacterial polymersome-based wound dressing spray, oil based bactericides, and oil based virucides.

The method of any other embodiment, wherein the at least one scaffolding element is selected from the group consisting of bioprinted skin tissue, a bioprinted gel configured for application to a skin wound, bioprinted muscle tissue, bioprinted bone tissue, bioprinted nerve tissue, and bioprinted connective tissue.

The method of any other embodiment, further comprising the step of adding at least one tissue regeneration factor to the bioactive suspension; wherein the step of adding at least one tissue regeneration factor to the bioactive suspension immediately follows the step of separating the tissue cells from the initial cell suspension.

The method of any other embodiment, further comprising the step of adding at least one efficacious element to the bioactive suspension.

The method of any other embodiment, wherein the tissue sample comprises human skin.

The method of any other embodiment, wherein the human skin comprises split-thickness human skin.

The method of any other embodiment, wherein the human skin comprises epidermis.

The method of any other embodiment, wherein the human skin comprises epidermis and an epidermal-dermal junction.

The method of any other embodiment, wherein the human skin comprises epidermis, an epidermal-dermal junction, and dermis.

The method of any other embodiment, wherein the tissue sample is selected from the group consisting of adipose tissue and mesenchymal skin tissue.

The method of any other embodiment, wherein the tissue sample comprises non-human skin tissue.

The method of any other embodiment, wherein the tissue sample is selected from the group consisting of urodele skin tissue, anuran skin tissue, porcine skin tissue, bovine skin tissue, murine skin tissue, and marsupial skin tissue.

The method of any other embodiment, wherein the tissue sample is autologous relative to the patient.

The method of any other embodiment, wherein the tissue sample is allogeneic relative to the patient.

The method of any other embodiment, wherein the tissue sample is xenogeneic relative to the patient.

The method of any other embodiment, wherein the treatment cells are selected from the group consisting of tissue cells, wild-type allogeneic cells, wild-type xenogeneic cells, cultured cells, and gene-edited cells.

The method of any other embodiment, wherein the gene-edited cells are selected from the group consisting of epidermolysis bullosa-corrected cells, reverse-differentiated cells, and forward-differentiated cells.

The method of any other embodiment, wherein the forward-differentiated cells are selected from the group consisting of somatic cells with lengthened telomeres and cells that express telomerase.

The method of any other embodiment, wherein the forward-differentiated cells express telomerase.

The method of any other embodiment, wherein the at least one period of enzymatic disaggregation time is at least 30 minutes.

The method of any other embodiment, wherein the at least one tissue regeneration factor is released by an epidermal cell.

The method of any other embodiment, wherein the at least one tissue regeneration factor is released by an epithelial cell.

The method of any other embodiment, wherein the at least one tissue regeneration factor is released by a cell from at least one area of an epidermal-dermal junction selected from the group comprising the basal cell plasma membrane, lamina lucida, basal lamina, or sub-basal lamina fibrous component area.

The method of any other embodiment, wherein the at least one tissue regeneration factor is selected from the group consisting of heat shock protein HSP90, epidermal growth factor (EGF), brain-derived neurotrophic factor (BDNF), interleukin-33 (IL-33), macrophage colony-stimulating factor (MCSF), stem cell factor (SCF), growth hormone (GH), insulin-like growth factor binding protein 1 (IGFBP-1), heparin-binding epidermal growth factor-like growth factor (HB-EGF), amphiregulin (AR), interleukin 1 alpha (IL-1α), vascular endothelial growth factor (VEGF), transforming growth factor alpha (TGF-α), vascular endothelial growth factor receptor 2 (VEGFR2), tumor necrosis factor receptor 2 (TNF RII), basic fibroblast growth factor (bFGF), interleukin-1 receptor antagonist (IL-1ra), tissue inhibitor matrix metalloproteinase 1 (TIMP1), stem cell factor receptor (SCF R), fibroblast growth factor 4 (FGF4), tumor necrosis factor receptor 1 (TNFR1), intercellular adhesion molecule 1 (ICAM-1), cathelicidin peptide LL-37, macrophage colony-stimulating factor receptor (MCSF R), hepatocyte growth factor (HGF), epidermal growth factor receptor (EGFR), antithrombin III (ATIII), tissue inhibitor matrix metalloproteinase 2 (TIP-2), and high mobility group box protein 1 (HMGB1).

The method of any other embodiments, wherein the at least one efficacious element is selected from the group consisting of hyaluronic acid, heat shock protein(s), platelet-enriched plasma, adipose stem cells, mesenchymal stem cells, a phytoconstituent, a trace element, a JAK inhibitor, an enzyme deactivation element, or one or more tissue regeneration factors supplied from a source other than the tissue sample used to make the bioactive suspension.

The method of any other embodiment, wherein the at least one tissue regeneration factor is present at <20 pg/ml of the bioactive suspension.

The method of any other embodiment, wherein the at least one tissue regeneration factor is present at 20-500 pg/ml of the bioactive suspension.

The method of any other embodiment, wherein the at least one tissue regeneration factor is present at 500-1000 pg/ml of the bioactive suspension.

The method of any other embodiment, wherein the at least one tissue regeneration factor is present at 1,000-10,000 pg/ml of the bioactive suspension.

The method of any other embodiment, wherein the at least one tissue regeneration factor is present at 10,000-100,000 pg/ml of the bioactive suspension.

The method of any other embodiment, wherein the at least one tissue regeneration factor is present at 100,000-500,000 pg/ml of the bioactive suspension.

The method of any other embodiment, wherein the at least one tissue regeneration factor is present at >500,000 pg/ml of the bioactive suspension.

The method of any other embodiment, wherein the at least one tissue regeneration factor is antithrombin III or tissue inhibitor matrix metalloproteinase 2.

The method of any other embodiment, wherein the at least one tissue regeneration factor is selected from the group consisting of intercellular adhesion molecule 1, cathelicidin peptide LL-37, macrophage colony-stimulating factor receptor, hepatocyte growth factor, and epidermal growth factor receptor.

The method of any other embodiment, wherein the at least one tissue regeneration factor is selected from the group consisting of epidermal growth factor, brain-derived neurotrophic factor, interleukin-33, macrophage colony-stimulating factor, stem cell factor, growth hormone, insulin-like growth factor binding protein 1, heparin-binding epidermal growth factor-like growth factor, and amphiregulin.

The method of any other embodiment, wherein the at least one tissue regeneration factor is selected from the group consisting of interleukin 1 alpha, vascular endothelial growth factor, transforming growth factor alpha, vascular endothelial growth factor receptor 2, tumor necrosis factor receptor 2, basic fibroblast growth factor, interleukin-1 receptor antagonist, tissue inhibitor matrix metalloproteinase 1, stem cell factor receptor, fibroblast growth factor 4, and tumor necrosis factor receptor 1.

The method of any other embodiment, wherein the enzyme solution comprises at least one enzyme in solution, and wherein the at least one enzyme is selected from the group consisting of trypsin, dispase, collagenase, trypsin-EDTA, thermolysin, pronase, hyaluronidase, elastase, papain and pancreatin. In some variations, one or more enzymes such as trypsin, dispase, collagenase, trypsin-EDTA, thermolysin, pronase, hyaluronidase, elastase, papain and pancreatin.

The method of any other embodiment, wherein the enzyme solution is heated to a temperature ranging from between about 30° C. and about 37° C.

The method of any other embodiment, wherein the at least one period of enzymatic disaggregation time ranges from one minute to sixty minutes.

The method of any other embodiment, wherein the at least one period of enzymatic disaggregation time ranges from 15 minutes to thirty minutes.

The method of any other embodiment, wherein the step of mechanically disaggregating the tissue sample comprises one or more of scraping the tissue sample with a scalpel, grinding the tissue sample with a pestle, or slicing the tissue sample with a blade.

The method of any other embodiment, wherein the at least one cell separation procedure comprises a filter or a centrifuge.

The method of any other embodiment, wherein the centrifuge is selected from the group comprising a differential pelleting centrifuge, an analytical ultracentrifuge (AUC), a preparative ultracentrifuge, and a density gradient centrifuge.

The method of any other embodiment, wherein the filter size is between 50 μm and 200 μm.

The method of any other embodiment, wherein the filter size is between 75 μm and 150 μm.

The method of any other embodiment, wherein the filter size is 100 μm.

The method of any other embodiment, wherein the filter size is 50 μm.

The method of any other embodiment, wherein the step of applying the bioactive suspension to at least one treatment site comprises one or more of spraying or dripping the bioactive suspension to the at least one treatment site.

The method of any other embodiment, wherein the at least one treatment site is a wound, wherein the wound is selected from the group consisting of an excised wound, a chronic wound, and a debrided wound.

The method of any other embodiment, wherein the at least one treatment site is a skin disorder, wherein the skin disorder is selected from the group consisting of vitiligo and epidermolysis bullosa.

The method of any other embodiment, wherein the at least one treatment cell is one or more cell types selected from the group consisting of cells obtained from the freshly disaggregated tissue used to obtain the bioactive suspension, cultured cells, gene-edited cells, induced pluripotent stem cells (iPSCs), and forward-differentiated cells.

The method of any other embodiment, wherein the at least one efficacious element comprises at least one phytoconstituent.

The method of any other embodiment, wherein the at least one phytoconstituent is selected from the group consisting of alkaloids, steroids, sterols, polyphenols, triterpenes, glycosides, carbohydrates, proteins, amino acids, flavonoids, saponins, phenolic compounds, and tannins.

The method of any other embodiment, wherein the at least one phytoconstituent is derived from a source selected from the group consisting of Celosia argentea, Buddleja globosa, Onosma argentatum, Scrophularia nodosa, Chromolaena odorata, Combretum smeathmanni, Phyllanthus muellerianus, Pycnanthus angolensis, Aloe vera, Alternanthera brasiliana, Dalbergia odorifera, Epimedium sagittatum, Trichosanthes kirilowii, Allamanda cathartica, Anogeissus latifolia, Carallia brachiate, Cynodon dactylon, Allium cepa, Areca catechu, Berberis lyceum, Carapa guainensis, Carica papaya, Acacia nilotica, Aspilia Africana, Emblica officinalis, Memecylon umbellatum, Rubia cordifolia, Achillea millefolium, Centella asiatica, Cocos nucifera, Crocus sativus, Cannabis sativa, Cannabis indica, Cannabis ruderalis, Laurus nobilis, Kaempferia galangal, Cecropia pelfata, Lawsonia inormis, Radix paeoniae, Lycopodium serratum, Jasminum grandiflorum, Bambusa vulgaris, Utleria solicifolia curcas, Clerodendrum infortunatu, Centella asiatica, Comphora officinarum, Shorea robusta, Apis mellifera, Sesamum indicum, Azardica indica, Vitex nigundo, Emblica officinalis (in some embodiments, the bark) Gaertn, Tridox procumbens, Arctium lappa, Astragalus propinquus, Rehmannia glutinosa, Ampelopsis japonica, Andrographis paniculata, Angelica sinensis, Blumea balsamifera, Boswellia sacra, Agrimonia eupatoria, Nelumbo nucifera, Boswellia sacra, pollen from Typha angustifoliae, Caesalpinia sappan, Calendula officinalis, Camellia sinensis, Carthamus tinctorius, Celosia argentea, Centella asiatica, Cinnamomum cassia, Commiphora myrrha, Curcuma longa, Daphne genkwa, Entada phaseoloides, Hibiscus rosa-sinensis, Ganoderma lucidum, Ligusticum striatum, Lonicera japonica, Paeonia suffruticosa, Panax ginseng, Panax notoginseng, Polygonum cuspidatum, Lithospermum erythrorhizon, Rheum officinale, Rhodiola imbricata, Salvia miltiorrhiza, Sanguisorba officinalis, Sophora flavescens, Stemona tuberosa, Wedelia trilobata, Zanthoxylum bungeanum, common lavender, chamomile, echinacea, rosemary, hemp, turmeric, orange flower, jasmine, avocado, and flaxseed, as well as any plant known in the art to provide one or more compounds that accelerate the healing process. In some embodiments, the at least one phytoconstituent may comprise a compound derived from an essential oil selected from the group comprising African lemon bush (Lippia javanica) oil, anise oil, bay oil, bergamot oil, boronia oil, cannabis oil, canola oil, carrot oil, cassia oil, catnip oil, cedarwood oil, chamomile oil, cinnamon oil, citronella oil, clary sage oil, clove oil, cypress oil, eucalyptus oil, galbanum oil, garlic oil, ginger oil, geranium oil, grapefruit oil, hazelnut oil, hemp oil, jasmine oil, jojoba oil, lavender oil, lavandin oil, lemon oil, lime oil, mandarin oil, nutmeg oil, orange oil, palma rosa oil, patchouli oil, Peru balsams, peppermint oil, rosemary oil, rosewood oil, sage oil, sandalwood oil, spear mint oil, star anise oil, tea tree oil, tangerine oil, thyme oil, tolu, verbena oil, white clover oil, ylang ylang oil.

The method of any other embodiment, wherein the at least one efficacious element comprises at least one trace element.

The method of any other embodiment, wherein the at least one trace element is selected from the group consisting of iron, manganese, zinc, copper, magnesium, nickel, boron, or molybdenum, or one or more vitamins selected from the group comprising vitamin A, vitamin B6, vitamin B12, vitamin C, vitamin D, vitamin E, folic acid, K, niacin, calcium, phosphorus, potassium, sodium, chlorine, sulfur, fluoride, vanadium, nitrogen, chromium, iodine, and selenium.

The method of any other embodiment, wherein the at least one efficacious element comprises at least one Janus kinase inhibitor (JAK inhibitor).

The method of any other embodiment, wherein the at least one Janus kinase inhibitor (JAK inhibitor) is selected from the group consisting of Abrocitinib, Delgocitinib, Fedratinib, Ruxolitinib, Upadacitinib, Tofacitinib, Baricitinib, Peficitinib, Filgotinib, Fedratinib, Oclacitinib, Cerdulatinib, Itacitinib, Gandotinib, Lestaurtinib, Momelotinib, Pacritinib, Deucravacitinib, Cucurbitacin I, CHZ868, SHR0302, and BMS-986165.

The method of any other embodiment, wherein the at least one Janus kinase inhibitor (JAK inhibitor) is an extract of a plant selected from the group consisting of blackberry, boysenberry, feijoa, pomegranate, rosehip and strawberry.

The method of any other embodiment, wherein the at least one Janus kinase inhibitor (JAK inhibitor) is selected from the group consisting of Castalagin, Castalin, Casuarictin, Grandinin, Roburin A, Tellimagrandin II, Terflavin B, Vescalagin, Pomegranate ellagitannins, Punicalagin, and Punicalin.

The method of any other embodiment, wherein the at least one efficacious element is at least one enzyme inhibitor.

The method of any other embodiment, wherein the at least one enzyme inhibitor is selected from the group consisting of a protease inhibitor, carbohydrase inhibitor, lipase inhibitor, oxidoreductase inhibitor, transferase inhibitor, hydrolase inhibitor, lyase inhibitor, isomerase inhibitor, ligase inhibitor, matrix metalloproteinases, and translocase inhibitor.

The method of any other embodiment, wherein the at least one efficacious element is at least one antibacterial agent.

The method of any other embodiment, wherein the at least one antibacterial agent is selected from the group consisting of ciprofloxacin, amoxicillin-clavulanate, cephalexin, clindamycin, dicloxacillin, doxycycline, trimethoprim-sulfamethoxazole, honey, acetic acid, iodine, povidone iodine, cadexomer iodine, silver sulphadiazine (SSD), bacitracin, mafenide, mupirocin, neomycin, and curcumin.

A bioactive suspension produced by the process of any embodiment disclosed herein.

A bioactive suspension produced by the process of any embodiment disclosed herein further comprising the step of adding treatment cells.

A bioactive suspension produced by the process of any embodiment disclosed herein further comprising the step of adding at least one efficacious element.

A bioactive suspension produced by the process of any embodiment disclosed herein further comprising the steps of freezing and thawing the bioactive suspension.

A bioactive suspension produced by the process of any embodiment disclosed herein further comprising the step of adding the bioactive suspension to at least one scaffolding element.

A synthetic bioactive suspension, comprising an effective amount of buffer and at least one added element selected from the group consisting of at least one tissue regeneration factor and at least one efficacious element.

A synthetic bioactive suspension made according to any embodiment, further comprising at least one treatment cell.

A synthetic bioactive suspension made according to any embodiment, wherein the at least one tissue regeneration factor is at least five tissue regeneration factors selected from the group consisting of epidermal growth factor (EGF), stem cell factor (SCF), heparin-binding epidermal growth factor-like growth factor (HB-EGF), amphiregulin (AR), vascular endothelial growth factor (VEGF), transforming growth factor alpha (TGF-α), vascular endothelial growth factor receptor 2 (VEGFR2), fibroblast growth factor 4 (FGF-4), and hepatocyte growth factor (HGF).

A synthetic bioactive suspension made according to any embodiment, wherein the at least one tissue regeneration factor is high mobility group box protein 1 (HMGB1), and wherein high mobility group box protein 1 (HMGB1) is present at least at 500,000 pg/ml.

A bioactive suspension combination product, comprising a bioactive suspension made according to any method disclosed herein and at least one scaffolding element.

A synthetic bioactive suspension combination product, comprising a synthetic bioactive suspension made according to any method disclosed herein and at least one scaffolding element.

What is claimed is:

1. A synthetic bioactive suspension, comprising:
an effective amount of buffer; and
at least one added element selected from the group consisting of at least one tissue regeneration factor and at least one efficacious element.

2. The synthetic bioactive suspension of claim 1, wherein the at least one tissue regeneration factor is at least five tissue regeneration factors selected from the group consisting of epidermal growth factor (EGF), stem cell factor (SCF), heparin-binding epidermal growth factor-like growth factor (HB-EGF), amphiregulin (AR), vascular endothelial growth factor (VEGF), transforming growth factor alpha (TGF-α), vascular endothelial growth factor receptor 2 (VEGFR2), fibroblast growth factor 4 (FGF-4), and hepatocyte growth factor (HGF).

3. The synthetic bioactive suspension of claim 1, wherein the at least one tissue regeneration factor is high mobility group box protein 1 (HMGB1), and wherein high mobility group box protein 1 (HMGB1) is present at least at 500,000 pg/ml.

4. The synthetic bioactive suspension of claim 1, wherein the at least one efficacious element is at least one phytoconstituent.

5. The synthetic bioactive suspension of claim 1, wherein the at least one efficacious element is at least one trace element.

6. The synthetic bioactive suspension of claim 1, wherein the at least one efficacious element is at least one Janus kinase inhibitor.

7. The synthetic bioactive suspension of claim 1, wherein the at least one efficacious element is at least one enzyme inhibitor.

8. The synthetic bioactive suspension of claim 1, wherein the at least one efficacious element is at least one antibacterial agent.

9. The synthetic bioactive suspension of claim 1, further comprising at least one treatment cell.
